(19) United States
(12) Patent Application Publication
(10) Pub. No.: US 2022/0409351 A1
(43) Pub. Date: Dec. 29, 2022

(54) ORAL APPLIANCE FOR DELIVERY OF AN ANTIMICROBIAL COMPOSITION

(71) Applicant: Emanate Biomedical, Inc., New York, NY (US)

(72) Inventors: Peter John Zegarelli, Sleepy Hollow, NY (US); Jarret Fass, New York, NY (US)

(73) Assignee: Emanate Biomedical, Inc., New York, NY (US)

(21) Appl. No.: 17/778,564

(22) PCT Filed: Nov. 20, 2020
(86) PCT No.: PCT/US20/61500

§ 371 Date: May 20, 2022

Related U.S. Application Data
(60) Provisional application No. 62/939,249, filed on Nov. 22, 2019.

(51) Int. Cl. A61C 19/06; A61K 31/155; A61K 9/06; A61K 47/32

(52) U.S. Cl. CPC A61C 19/063; A61K 31/155; A61K 9/06; A61K 47/32

(57) ABSTRACT

An oral appliance, a method of making the oral appliance for delivering an antimicrobial composition to an oral cavity and a method of treatment are provided. The oral appliance has an exterior surface and an interior surface, the interior surface of the oral appliance configured to contour at least a portion of teeth and/or soft tissue areas of the oral cavity. The antimicrobial is dispensed by itself or as a composition in a carrier at discrete regions of the interior surface, the exterior surface or both the interior surface and the exterior surface of the oral appliance for delivering the antimicrobial or an antimicrobial composition to the oral cavity, wherein the antimicrobial is present in the carrier in an amount of about 0.01% to about 20% w/w, v/v or w/v based on a total weight or a total volume of the carrier.

### BACKGROUND

The present disclosure is generally related to the field of oral appliances for delivering an antimicrobial composition to the oral cavity.

Dental plaque is a precursor of calculus. Plaque is recognized as a precursor of such oral diseases as caries and gum disease. Gum disease (gingivitis and periodontitis) is an infectious disease characterized by inflammation of the gums in response to a build-up of pathological bacteria around the teeth and gums. The bacteria associated with plaque can secrete enzymes and endotoxins which can irritate the gums and cause gingivitis. As the gums become increasingly irritated by this process they tend to bleed, lose their toughness and resiliency, and separate from the teeth, leaving periodontal pockets in which debris, secretions, more bacteria and toxins further accumulate. In periodontitis, the gums recede as the periodontal ligament is damaged. This leads to the formation of periodontal pockets. These pockets host a higher percentage of pathologic anaerobic bacteria that cause more inflammation than in healthy gums. The “red complex” bacteria found in periodontal pockets are inflammophillic—that is, they feed off of the inflammatory tissue breakdown products. This dysbiosis, or imbalance in relative abundance or influence of pathologic bacteria, becomes self-perpetuating in a positive feedback loop. Eventually, the degradation of the supporting structures of the teeth, including alveolar bone, leads to bone loss, which when severe can lead to the eventual loss of the tooth.

In treating gum disease, consideration should be given to treat the infection. Current treatment of periodontal disease consists of scaling and root planning, which mechanically removes the bacteria from the pockets and smooths the surface of the root of the tooth to help prevent bacteria from building up. Patients cannot access deep pockets easily, so are instructed to return to the periodontist for routine periodontal maintenance therapy, on average every 3 to 6 months.

Chlorhexidine rinse is often prescribed as an adjunct to scaling and root planning It is a broad-spectrum antimicrobial that can be effective against pathologic red complex bacteria. Often times, for chlorhexidine to be effective, it can have a minimum inhibitory concentration (MIC) when the antimicrobial is in direct contact with the bacteria. This is the concentration at which the bacteria's growth is inhibited. Minimum bactericidal concentration (MBC) is the concentration at which the bacteria are irreversibly killed, after a qualified amount of time. Chlorhexidine's MIC against oral pathogenic bacteria is 0.0125% w/v, and its MBC is 0.5% w/v at 10 minutes. By inhibiting the growth of bacteria, the host is able to recover and heal, breaking the pathogenic cycle. Because of chlorhexidine's cationic binding properties, a 0.12% w/v oral rinse maintains MIC for 4 hours after a 30 second rinse. It does not reach MIC concentrations subgingivally and causes other local adverse effects such as stained teeth, unpleasant taste and black tongue. There is little proven clinical benefit of the chlorhexidine rinse for periodontitis.

Oral chlorhexidine treatments, however, suffer from additional drawbacks. For example, it is well known that chlorhexidine products have an extremely bitter taste. This objectionable taste causes major compliance problems, particularly with pediatric patients. In order to combat the bitter taste, certain flavoring agents and sweetening agents have been added to chlorhexidine formulations. Additionally, chlorhexidine is a strong basic material that reacts with a wide variety of compounds and chemical structures that are often used in the commercial production of chlorhexidine products. For example, the addition of many flavoring agents, which are often aldehydes in structure, or sweetening agents can reduce or eliminate the antimicrobial activity of chlorhexidine via chemical reactions that include salt formation and precipitation.

There is therefore a clinical need for an easy-to-use oral appliance that can be used at home, that treats periodontal pockets effectively with an antimicrobial and is effective at overcoming one or more of the problems associated with existing antimicrobial rinses. It would also be beneficial to provide oral appliances that can be easily loaded with, for example, chlorhexidine compositions at discrete regions of an oral appliance to control its delivery at the target tissue site within the oral cavity.

### SUMMARY

Oral appliances for delivering an antimicrobial or an antimicrobial composition to an oral cavity are provided. The oral appliances have an exterior surface and an interior surface, the interior surface of the oral appliance configured to contour at least a portion of teeth and/or soft tissue areas of the oral cavity. The antimicrobial is dispensed by itself or as a composition in a carrier at discrete regions of the interior surface, the exterior surface or both the interior surface and the exterior surface of the oral appliance for delivering the antimicrobial or antimicrobial composition to the oral cavity, wherein the antimicrobial is present in the carrier in an amount of about 0.01% to about 20% w/w, v/v or w/v based on a total weight or a total volume of the carrier. In some embodiments, the carrier can be a hydrogel containing the antimicrobial. The antimicrobial (e.g., chlorhexidine) can be disposed uniformly throughout the hydrogel or be disposed at discrete regions of the hydrogel.

In various embodiments, a method of making an oral appliance for delivering an antimicrobial or antimicrobial composition to an oral cavity is also provided. The method comprises providing an oral appliance having an exterior surface and an interior surface, the interior surface of the oral appliance configured to contour at least a portion of teeth and/or soft tissue areas of the oral cavity; and dispensing the antimicrobial or antimicrobial composition in a carrier at discrete regions of the interior surface, the exterior surface or both the interior surface and the exterior surface of the oral appliance for delivering the antimicrobial or the antimicrobial composition to the oral cavity, wherein the antimicrobial is present in the carrier in an amount of about 0.01% to about 20% w/w, v/v or w/v based on a total weight or a total volume of the carrier.

A method of treating an infected tissue of an oral cavity using the oral appliance of this disclosure is also provided. The method comprises providing an oral appliance for delivering an antimicrobial in a carrier to the infected tissue of the oral cavity, the oral appliance having an exterior surface and an interior surface, the interior surface configured to contour at least a portion of teeth and/or soft tissue areas of the oral cavity, the interior surface of the oral appliance having the antimicrobial in a carrier disposed at a discrete regions of the interior surface, the exterior surface or both the interior and exterior surface of the oral appliance for delivering the antimicrobial or antimicrobial composition to the infected tissue, wherein the antimicrobial is present in the carrier in an amount of about 0.01% to about 20% w/w, v/v or w/v based on a total weight or a total volume of the carrier. In some embodiments, the infected tissue comprises a subgingival space, a gingival crevice or periodontal pocket. In other aspects, the infected tissue is periodontal tissue. In various embodiments, the oral appliance allows for the precise dosing of the antimicrobial composition to a particular amount of surface area of the infected tissue.

An antimicrobial composition for an oral appliance is also provided. The antimicrobial composition comprises chlorhexidine in a carrier, the chlorhexidine in an amount of about 0.01% to about 20% w/w, v/v or w/v based on a total weight or a total volume of the composition, and the carrier comprising at least one of hydroxyethlymethacrylate (HEMA), polyvinyl alcohol (PVA), ethylene glycol dimethacrylate (EGDMA), hydroxypropylcellulose (HPC) or poly(2-hydroxyethyl methacrylate) (pHEMA) or a combination thereof in an amount of from about 99.5% to about 80% w/w, v/v or w/v based on a total weight or a total volume of the composition. In some embodiments, the antimicrobial composition can be mixed with PVA which allows the hydrogel to be preloaded with a precise amount of antimicrobial composition. Thus, the antimicrobial composition can be precisely dosed before dispensing it into the oral appliance.

Additional features and advantages of various embodiments will be set forth in part in the description that follows, and in part will be apparent from the description, or may be learned by practice of various embodiments. The objectives and other advantages of various embodiments will be realized and attained by means of the elements and combinations particularly pointed out in the description and appended claims.

### BRIEF DESCRIPTION OF THE FIGURES

In part, other aspects, features, benefits and advantages of the embodiments will be apparent with regard to the following description, appended claims and accompanying drawings.

FIG. 1 illustrates an enlarged side view of an embodiment of the oral appliance configured to contour at least a portion of the teeth and/or soft tissues of a patient, the oral appliance is shown without teeth and/or soft tissues inserted in the oral appliance.

FIG. 2 illustrates an enlarged side view of an embodiment of the oral appliance, where the antimicrobial is shown disposed in a carrier, which is a polymer (e.g., hydrogel) that is adjacent to an infected tissue, which is shown as the gingival sulcus region. This view has the teeth and gums loaded in the interior of the oral appliance and the antimicrobial is disposed in a polymer at a discrete region adjacent to the infected tissue of the oral cavity. It will be understood by those of ordinary skill in the art that the antimicrobial can be disposed uniformly throughout the carrier or at a discrete region of the carrier depending on where the treatment area.

FIG. 3A illustrates an enlarged view of the carrier, which is a polymer (e.g., hydrogel) containing the antimicrobial, which will be at a discrete region along the sulcus (gumline). The antimicrobial is disposed at discrete regions of the carrier.

FIG. 3B illustrates an enlarged interior view of an image of the oral appliance having antimicrobial disposed at a discrete region of the carrier (e.g., hydrogel) targeting the sulcus. The hydrogel shown in FIG. 3A is disposed within the oral appliance in FIG. 3B.

FIG. 4 illustrates an enlarged cross-sectional view of the anatomy of the gums and a tooth including free gingiva, attached gingiva, lining mucosa, the periodontal pocket or crevice, the cementoenamel junction (CEJ), periodontal ligament, cementum, the enamel, dentin, pulp and the junctional epithelium. In some embodiments, the periodontal pocket is targeted for delivery. The design of the antimicrobial composition of the oral appliance is to target the periodontal pocket or crevice of the sulcus and extrude the antimicrobial into the entrance of the periodontal pocket.

FIG. 5 illustrates an enlarged cross-sectional view of a portion of the oral appliance 400. In the embodiment shown, antimicrobial is disposed in a porous material that is a hydrogel at a discrete region of the oral appliance. The hydrogel is shown in an uncompressed state and when worn with slight pressure, the hydrogel will be compressed against, among other things, the gingival crevice or periodontal pocket causing a seal of the entrance of the gingival crevice or periodontal pocket, which prevents oral fluids (e.g., saliva, exudate, or other captured foreign fluids from insertion of the device, etc.) from entering the crevice or pocket, which allows release of the antimicrobial in the gingival crevice or periodontal pocket and allows the hydrogel to absorb or wick fluid from the crevice or pocket.

FIG. 6 illustrates an enlarged cross-sectional view of a portion of the oral appliance that is placed adjacent to the teeth and gums. In the embodiment shown, antimicrobial is disposed in a porous material that is a hydrogel at a discrete region of the oral appliance. The hydrogel is shown in a compressed state, where the device is worn and the hydrogel is compressed against, among other things, the gingival crevice or periodontal pocket causing a seal or encapsulation of the entrance of the gingival crevice or periodontal pocket, which prevents oral fluids (e.g., saliva, exudate, or other captured fluids upon insertion of the device, etc.) from entering the crevice or pocket. The hydrogel allows release of the antimicrobial into the gingival crevice or periodontal pocket to treat the inflamed tissue shown by the down arrows. The hydrogel also absorbs or wicks oral fluids from the crevice or pocket, which aides healing, shown by the up arrows.

FIG. 7 illustrates overlaid UV visible scans of carrier (e.g., hydrogel) reactants including EGDMA, HEMA, pHEMA and the photoinitiator Irgacure.

FIG. 7A illustrates a UV visible scan of 50% pHEMA 300kDa as prepared in Example 10 of this disclosure.

FIG. 8A is a graph generated by a TA.TX Plus texture analyzer for a 3% HPC 1.15M polymer as prepared in Example 9 of this disclosure.

FIG. 8B is a graph generated by a TA.TX Plus texture analyzer for a hydrogel of 50% pHEMA 300k Da as prepared in Example 10 of this disclosure.

FIG. 9 shows pictures of hydrogel samples taken in loading and release assays.

FIG. 10A shows pictures of 15% pHEMA hydrogel sample prepared as in Example 7 and a 3% HPC polymer sample prepared as in Example 17 during treatment with 5% chlorhexidine gluconate for a loading and release sample post sonication treatment.

FIG. 10B shows a picture of a square piece of 0.75 rigid base material in 10 mL of artificial saliva after treatment with 5% chlorhexidine gluconate.

It is to be understood that the figures are not drawn to scale. Further, the relationship between objects in a figure may not be to scale and may in fact have a reverse relationship as to size. The figures are intended to bring understanding and clarity to the structure of each object shown, and thus, some features may be exaggerated in order to illustrate a specific feature of a structure.

### DETAILED DESCRIPTION

For the purposes of this specification and appended claims, unless otherwise indicated, all numbers expressing quantities of ingredients, percentages or proportions of materials, reaction conditions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges subsumed therein. For example, a range of “1 to 10” includes any and all subranges between (and including) the minimum value of 1 and the maximum value of 10, that is, any and all subranges having a minimum value of equal to or greater than 1 and a maximum value of equal to or less than 10, e.g., 5.5 to 10.

It is noted that, as used in this specification and the appended claims, the singular forms “a,” “an,” and “the,” include plural referents unless expressly and unequivocally limited to one referent. Thus, for example, reference to “an antimicrobial” includes one, two, three or more antimicrobials.

A “carrier” is any substrate used in the process of delivering the antimicrobial. The carrier serves to improve the selectivity, effectiveness, and/or safety of antimicrobial administration. Carriers can be used to control the release of an antimicrobial. This can be accomplished either by slow release of the antimicrobial over a long period of time (typically diffusion) or by triggered release at the antimicrobial's target by some stimulus, such as, for example, changes in pH, application of heat, activation by light, etc. Suitable carriers include one or more polymers, such as for example, hydrogels.

The term “porous” as used herein, refers to a carrier which is permeable such that fluids are movable therethrough by way of pores or other passages. An example of a porous material is a hydrogel material, a cellulosic material, concrete, ceramics, foams, sponges and derivatives thereof. The porous material may be the result of using a low or high molecular weight polymer. In some embodiments, the polymer may be porous as it is dispensed at a low density on the oral appliance and/or substrate, or is dispensed in a geometric pattern, either as a specific structure or a randomized structure.

The term “non-porous” as used herein, refers to a material which is impermeable such that fluids cannot move through the material. The non-porous material may be the result of using a low or high molecular weight polymer. In some embodiments, the polymer may be non-porous as it is dispensed at a high density on the oral appliance and/or substrate in a solid form with no structural spacing to hold antimicrobials, as described above.

The term “hydrogel” or “hydrogels” refer to a broad class of polymeric materials, that may be natural or synthetic, which have an affinity for an aqueous medium, and are able to absorb aqueous medium, but which do not normally dissolve in the aqueous medium.

The term “antimicrobial” as used herein is generally refers to a group of medicaments that reduce, inhibit, or eliminate microbial growth. An antimicrobial includes an antibiotic, an antifungal, an antiprotozoal, an antiviral or a combination thereof. The term “antimicrobial” may be used interchangeably herein with the term “antimicrobial agent.” It will be understood that an “antimicrobial formulation” may include more than one antimicrobial agent, wherein exemplary combinations of antimicrobial agents include a combination of two or more antimicrobials.

The term “dental plaque” is a general term for the diverse microbial community (predominantly bacteria) found on the tooth surface, embedded in a matrix of polymers of bacterial and salivary origin.

The term “oral diseases” refers to diseases and disorders affecting the oral cavity or associated medical conditions. Oral diseases include, but are not limited to, inflammation, infection, dental caries, periodontal diseases (e.g., gingivitis, adult periodontitis, early-onset periodontitis, chronic periodontitis and/or aggressive periodontitis) or the like. Inflammatory diseases can also include benign and malignant tumors such as Lichen Planus and squamous cell carcinoma, respectively, as well as various yeast and fungal infections and conditions like Xerostomia.

The term “gingiva” or “gum” refers to a dense fibrous tissue and overlying mucous membrane enveloping alveolar processes of upper and lower jaws and surrounding the necks of teeth.

The term “gingivitis” refers to inflammation of gingival tissue without loss of connective tissue.

The term “inflamed tissue” refers to bone, teeth, or gingival tissue, that is red, swollen and can be painful. It will also more broadly include dental caries and/or hypersensitive areas of the teeth. Inflammation can be caused by trauma to the oral cavity, infection or other causes.

The term “periodontal disease” refers to an inflammatory process of the gingival tissues and/or periodontal membrane of the teeth, resulting in a deep gingival sulcus, possibly producing periodontal pockets and loss of alveolar bone.

The term “periodontitis” refers to inflammation and loss of connective tissue of the supporting or surrounding structure of teeth with loss of attachment.

The terms, “treating” or “treatment” includes “preventing” or “prevention” of disease. In addition, “treating” or “treatment” does not require complete alleviation of signs or symptoms, does not require a cure, and specifically includes protocols that have only a marginal effect on the patient.

The term “localized” delivery includes delivery where one or more antimicrobials contact the tooth and/or soft tissue areas, for example, the gingival margins of the teeth or a region inside of the mouth such as the palate, or in close proximity thereto.

The term “targeted delivery” includes delivery of one or more antimicrobials at the target site as needed for treatment of the disease or condition including cosmetic applications, for example, whitening teeth or removing stains. In some embodiments, the oral appliance can be used to deliver antimicrobial to the soft tissue of the inside of the mouth, including but not limited to any soft tissue adjacent or between the teeth, including but not limited to the papilla, tissue of the upper and lower dental arches, marginal gingiva, gingival sulcus, inter-dental gingiva, gingival gum structure on lingual and buccal surfaces up to and including the muco-gingival junction and/or the palate and/or the floor of the mouth. In various embodiments, the soft tissue area includes the muco-buccal folds, hard and soft palates, the tongue, lining mucosa, and/or attached gingival tissue.

The term “custom fit” as used herein, refers to an oral appliance that is specifically made via molding and/or 3D printing or other means, to correspond to at least a portion of a tooth, a selected number of teeth, all of the teeth and/or soft tissues found in the mouth of a specific individual patient. A custom fit oral appliance is not a generic device which is then heated or otherwise manipulated by a consumer, inserted into their mouth by themselves and then molded by that consumer to fit their own mouth. The patient image is the result of an action upon that particular individual by another person whereas the consumer is acting upon himself/herself by manually manipulating the generic material. In some embodiments, custom fit includes situations where the patient images himself or herself with a scanning device including those available in smartphone (e.g., I-phone, Android, Galaxy or the like) and then the appliance is made as a separate act.

Reference will now be made in detail to certain embodiments of the invention, examples of which are illustrated in the accompanying drawings. While the invention will be described in conjunction with the illustrated embodiments, it will be understood that they are not intended to limit the invention to those embodiments. On the contrary, the invention is intended to cover all alternatives, modifications, and equivalents, which may be included within the invention as defined by the appended claims.

The headings below are not meant to limit the disclosure in any way; embodiments under anyone heading may be used in conjunction with embodiments under any other heading.

### Oral Appliance

Numerous custom fit oral appliances can be made in a variety of ways including by traditional thermoforming, 3D printing or additive manufacturing, or injection molding or other ways. Unlike orthodontic appliances, the present oral appliance is not designed to move teeth and is not an orthodontic appliance. Therefore, a plurality of oral appliances will be configured to fit the teeth in the same position as was imaged within the oral appliance. The teeth position will not change. However, the antimicrobial disposed in or on the oral appliance will be in the same or different areas at different stages of the treatment regimen with a variety of oral appliances. Thus, kits containing a plurality of oral appliances can be provided with different treatment plans. For example, as the patient condition improves, each oral appliance will have a decreasing amount of antimicrobial or the antimicrobial can change as the treatment progresses.

In various embodiments, the oral appliance is monolithic or a single piece and the interior surface custom fit and formed to fit contours of the teeth and/or soft tissue areas inside the oral cavity of a patient in need of treatment. In this embodiment, the antimicrobial including the polymer is disposed at discrete regions of the oral appliance. The antimicrobial is part of the device and in some embodiments, the antimicrobial is not removable from it except by diffusion in the mouth. In certain embodiments, the oral appliance comprises, consists essentially of or consists of one, two, three, four, five or more oral appliances.

In various embodiments, the oral appliance is not monolithic or a single piece. The antimicrobial is disposed in a polymer, which is disposed in the interior or inside the oral appliance, but as a separate component to the oral appliance. For example, the antimicrobial can be disposed in porous material (e.g., polymer) that is configured to allow release of the antimicrobial when the oral appliance is worn.

In some embodiments, oral appliances include, but are not limited to, oral trays, oral holders, oral covers, or the like that are designed to be placed within the oral cavity. The interior surface and/or exterior surface of the oral appliance contains an antimicrobial disposed inside the porous portion of the polymer of the oral appliance and the antimicrobial can be disposed anywhere within or on the oral appliance as part of the device. In some embodiments, the exterior surface of the oral appliance is porous and allows antimicrobial to be released to adjacent teeth and/or soft or hard tissue, or into the mouth in general.

Numerous different oral appliances can be made by the methods of the present application, including custom fit oral appliances that correspond to a digital scan taken from the patient's mouth or impression molds. Custom fit oral appliances are generally described in U.S. Pat. No. 6,626,669 filed Apr. 15, 2002; U.S. Pat. No. 9,579,178 filed Jul. 12, 2013 and U.S. Pat. No. 9,649,182, to Peter J. Zegarelli, filed Jun. 18, 2015. The entire disclosure of this patent is herein incorporated by reference into the present disclosure.

The oral appliance when worn allows the interior and/or exterior surface of the oral appliance to be adjacent to the teeth and/or gums or other tissue in the oral cavity. In some embodiments, the oral appliance receives one or more teeth including one or more molars, premolars, incisors, cuspids, tooth implant, or combinations or portions thereof.

The contact of the oral appliance with the tissue, when the oral appliance contains an antimicrobial in the porous regions, will allow the antimicrobial to be released from the oral appliance to the target tissue areas in the oral cavity (e.g., gum, gum line, teeth, etc.) at the desired regions adjacent to the porous regions of the oral appliance. In this way, targeted therapy can be directed at the desired regions in the oral cavity. By providing an oral appliance with porous regions and non-porous regions, the antimicrobial release can be controlled to adjacent tissue or confined to those regions adjacent to the porous polymer with or without dilution by oral fluids such as saliva or releasing the antimicrobials onto non-dtargeted areas of the mouth with sometimes deleterious effects.

In some embodiments, the non-porous material is the structural backbone of the oral appliance and is present throughout the oral appliance to give it form, shape and structural integrity. The porous material parts (e.g., polymer) of the oral appliance are strategically placed about the oral appliance in order to deliver antimicrobials to those areas to be treated. These areas can be either internal or external to the oral appliance.

In some embodiments, the oral appliance is made from a porous material that contains the antimicrobial, and an agent that reduces porosity is applied to one or more discrete regions of the porous material to make the one or more discrete regions of the oral appliance non-porous as more particularly described in U.S. Patent Application No. 15/895,554 to Peter J. Zegarelli, filed on Feb. 13, 2018. The entire disclosure of this application is incorporated herein by reference into the present application. For example, a crosslinking agent can be used to reduce porosity of a porous oral appliance and make that region where the crosslinking agent is applied less porous to reduce or eliminate antimicrobial release from that region.

In some embodiments, the oral appliance can be made by controlling the print density of the polymer during 3D printing or additive manufacturing. For example, the same polymer can be printed (e.g., using the same print head) at a density of, for example, 0.25 g/cm³ to 0.5 g/cm³ at discrete regions to form the porous regions of the oral appliance and at a higher density for example, 0.8 g/cm³ to 1.5 g/cm³ to make the oral appliance non-porous at discrete regions.

In some embodiments, the oral appliance can be made by controlling the density of the polymer during 3D printing or additive manufacturing. For example, different polymers can be printed using two or more print heads, each print head having a different polymer. A high-density polymer can be used (e.g., 50,000 MW) and printed at discrete regions to form the non-porous regions of the oral appliance and another print head can use a low-density polymer (e.g., 5,000 MW) to make the oral appliance porous at discrete regions.

It will be understood that the oral appliance with discrete portions of the porous material and with discrete portions of non-porous material can be monolithic or a single piece having the same or different material. This type of oral appliance, in some embodiments, does not contain a porous insert after the oral appliance is made. Such porous inserts are described in U.S. Pat. No. 9,579,178, filed Jul. 12, 2013 to Peter J. Zegarelli. The entire disclosure of this patent is herein incorporated by reference into the present disclosure.

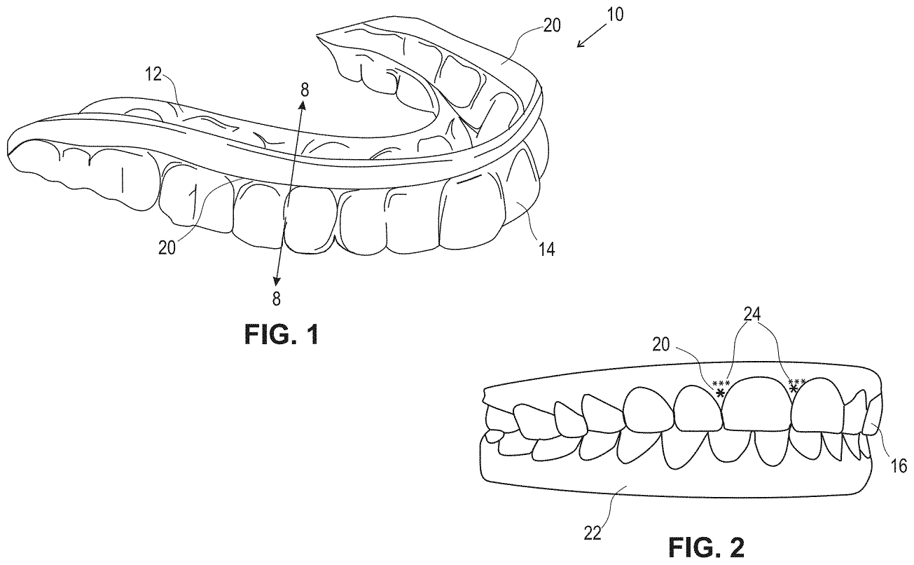

FIG. 1 is an enlarged side view of an embodiment of the oral appliance 10 before it is worn. The oral appliance has an interior surface 12 configured to contour at least a portion of the teeth and/or soft tissue areas of the oral cavity and an exterior surface 14, both comprising, in some embodiments, a polymer. The interior surface 12 is configured to contact at least all or a portion of one or more teeth and/or soft tissue areas of the oral cavity in a patient. The interior surface 12 is custom fit to the individual patient's mouth and configured to contour at least a portion of teeth and/or soft tissues of the oral cavity. In this view the interior surface 12 is configured to contact the teeth and soft tissue. Oral appliances include, but are not limited to, oral trays, oral holders, oral covers, or the like that are designed to be placed within the oral cavity. The interior surface 12 and/or exterior surface 14 of the oral appliance contains an antimicrobial disposed in or on the polymer (e.g., hydrogel) and the antimicrobial can be disposed anywhere within or on the oral appliance. For example, the antimicrobial can be disposed at discrete regions adjacent to the treatment area or uniformly disposed throughout the device. As the interior and/or exterior surface of the oral appliance contacts the oral cavity, the antimicrobial is released from the polymer (e.g., gel or hydrogel) by all or parts of the oral appliance contacting the desired treatment site or pressure from the device contacting tissue or fluid at the treatment site (e.g., gums, tissue, teeth, etc.).

In some embodiments, polymer containing antimicrobial can degrade over time by the action of enzymes, by hydrolytic action and/or by other similar mechanisms in the oral cavity. In some embodiments, all or discrete portions of the polymer containing an antimicrobial will degrade and release the antimicrobial at or near the target site in the oral cavity. The oral appliance will cover at least a portion of the teeth and or gums by applying the device over axis 8-8 to cover the area of the teeth and or gums, and the oral appliance will be adjacent to the gingival sulcus 20, which will allow the antimicrobial, if desired, to be released from the polymer to this area.

FIG. 2 illustrates an enlarged side view of an embodiment of the oral appliance, where the antimicrobial 24 is shown disposed in a carrier, which is a polymer (e.g., hydrogel) that is adjacent to an infected tissue, which is shown as the gingival sulcus region 20. This view has the teeth 16 and gums loaded in the interior of the oral appliance and the antimicrobial is disposed in a polymer at a discrete region adjacent to the infected tissue of the oral cavity. The exterior 22 of the oral appliance can be transparent or non-transparent. It will be understood by those of ordinary skill in the art that the antimicrobial can be disposed uniformly throughout the carrier or at a discrete region of the carrier depending on the location of the treatment area.

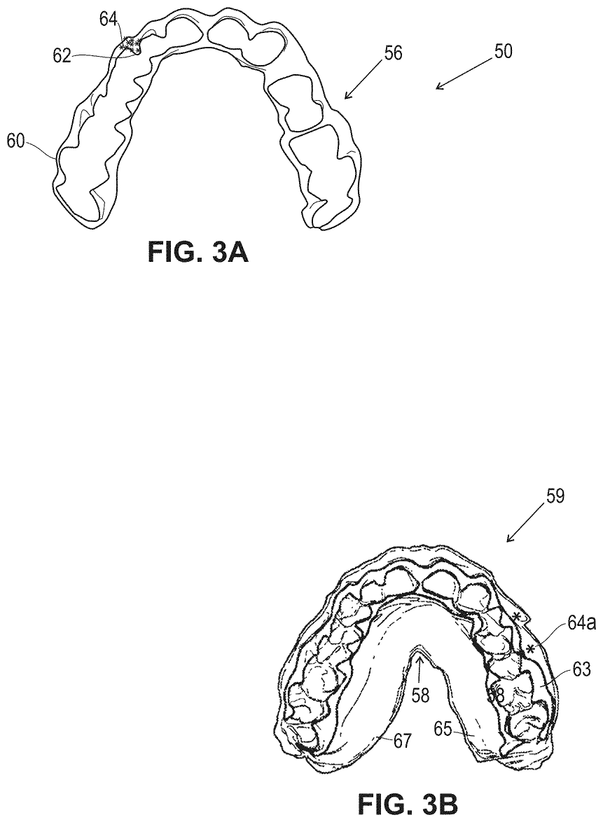

FIG. 3A illustrates an enlarged view of the oral appliance 50 having a carrier 60, which is a polymer (e.g., hydrogel) having a region containing the antimicrobial 64, which will be at a discrete region 62 adjacent to the sulcus 56. The antimicrobial is disposed at discrete regions of the carrier to allow treatment of infected tissue at the sulcus.

FIG. 3B illustrates an enlarged view of the oral appliance 59 that has regions along the sulcus (gumline) 63 where the antimicrobial 64a (e.g., chlorhexidine) will be loaded at discrete regions on the interior surface of oral appliance 59. The lower portion of the oral appliance corresponds to and will contact portions of the tongue and hard palate 65 as well as the soft palate 67 along each side of the oral cavity. The lower portion 58 of the oral appliance corresponds to and will contact portions of the tongue and hard palate 65 as well as the soft palate 67.

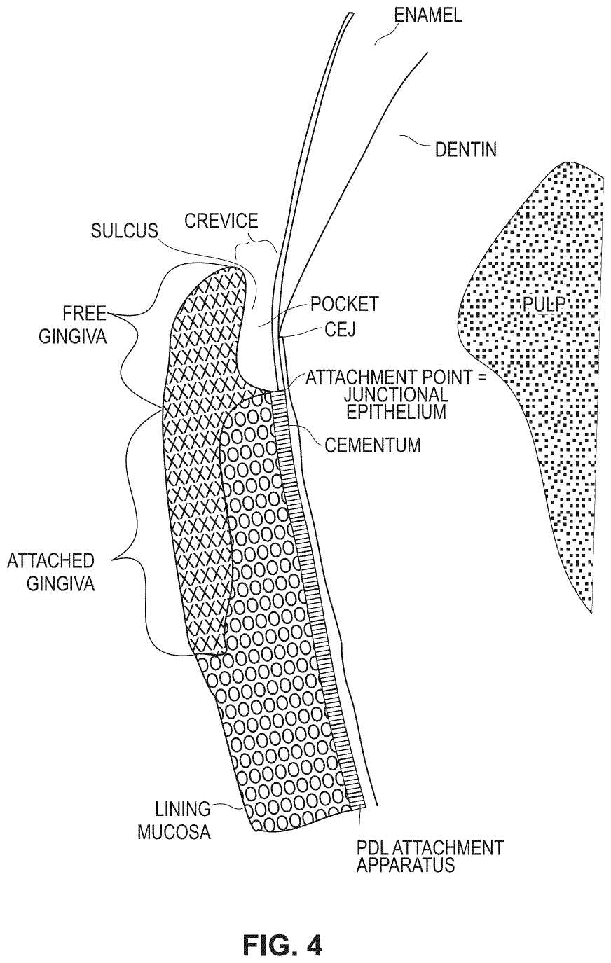

FIG. 4 illustrates an enlarged cross-sectional view of the anatomy of the gums and a tooth including free gingiva, attached gingiva, lining mucosa, the periodontal pocket or crevice, the cementoenamel junction (CEJ), periodontal ligament, cementum, the enamel, dentin, pulp and the junctional epithelium. In some embodiments, at the beginning of treatment, the top portion of the periodontal pocket is targeted for delivery of the antimicrobial in a top down approach.

In some embodiments, the oral appliance contains a carrier, which is a porous material (e.g., a hydrogel), which can contain an antimicrobial (e.g., chlorhexidine), which is released for a sustained period of time until the infection is reduced and is brought under greater control. The oral appliance described herein serves multiple purposes. It holds the antimicrobial in place with no or limited dilution by saliva or contamination by oral liquids, and it keeps antimicrobial at the top of the pocket. As the hydrogel is squeezed when the oral appliance is worn, the antimicrobial diffuses into the top of the pocket. The oral appliance design, characterized by the hydrogel placement over the gingival crevice, is akin to an encapsulation device, sealing off the opening of the pocket from outside contamination by saliva and other liquids, and forcing the hydrogel containing antimicrobial into the pocket entrance.

By encapsulating the gingival crevice, which is the entrance to the periodontal pocket, the oral appliance assures that the captured fluids, which are those fluids coating and surrounding the teeth and soft tissues when the tray is inserted, are pushed away from the crevice entrance and kept away by the encapsulating hydrogel over the crevice. Further, as the hydrogel is emptied of antimicrobial, Gingival Crevicular Fluids (GCFs) are wicked up and may be absorbed by the hydrogel in a fluid exchange, removing this contaminated exudate from the infected periodontium. GCFs include exudate fluids containing bacteria, dead cellular structures, interstitial fluids, inflammatory factors, etc. The greater the degree of inflammation due to the periodontal disease, the greater the rate of flow of the GCF. Removing the GCF from the pocket creates space for the antimicrobial and/or hydrogel to occupy. This will result in decreased inflammation and thus decreased GCF flow. As the gingiva at the top of the pocket begins to respond to the antimicrobial (e.g., antimicrobial), the surface inflammation will decrease, and the pocket will also shrink in depth. Once the antimicrobial treatment regimen has adequately reduced the pathologic microbiome, other avenues of treatment can be initiated to combat the other aspects of the periodontal disease sequence.

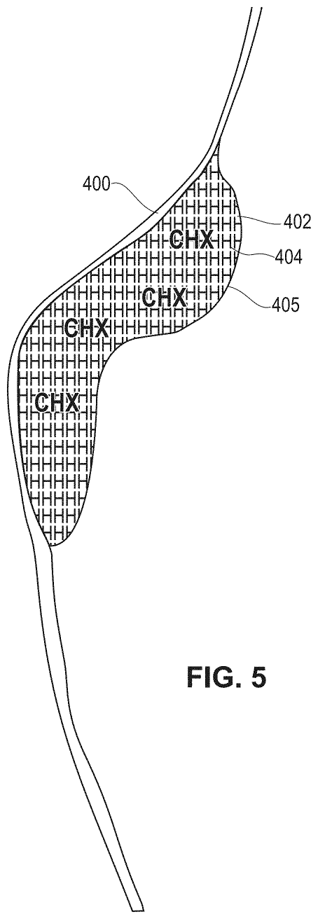

FIG. 5 illustrates an enlarged cross-sectional view of a portion of the oral appliance 400 that can be used in the top down approach as described in International application No. PCT/US20/59440 filed on Nov. 6, 2020, incorporated herein by reference in its entirety. In the embodiment shown, antimicrobial is disposed in a carrier, which is a porous material 402 containing an antimicrobial that is a hydrogel 404 at a discrete region of the oral appliance. The hydrogel is shown in an uncompressed state 405 and when worn with slight pressure, the hydrogel will be compressed against, among other things, the gingival crevice or periodontal pocket causing a seal of the entrance of the gingival crevice or periodontal pocket, which prevents other oral fluids (e.g., saliva, exudates, foreign liquids) from entering the crevice or pocket, which allows release of the antimicrobial in the gingival crevice or periodontal pocket and allows the hydrogel to absorb or wick fluid from the crevice or pocket. In this embodiment, the hydrogel is disposed at a discrete region of the oral appliance and is sized to be greater than the height, width, and length of the entrance of the periodontal pocket or crevice. In this way, when the device is worn, the hydrogel will contact the entrance of the periodontal pocket or crevice and encapsulate and seal it. In some embodiments, when the device is worn there will be a gap formed between the entrance of the periodontal pocket or crevice (top portion) and the bottom of the pocket by the junctional epithelium that will allow, among other things, antimicrobial to leach out of the hydrogel and treat the top of the periodontal pocket.

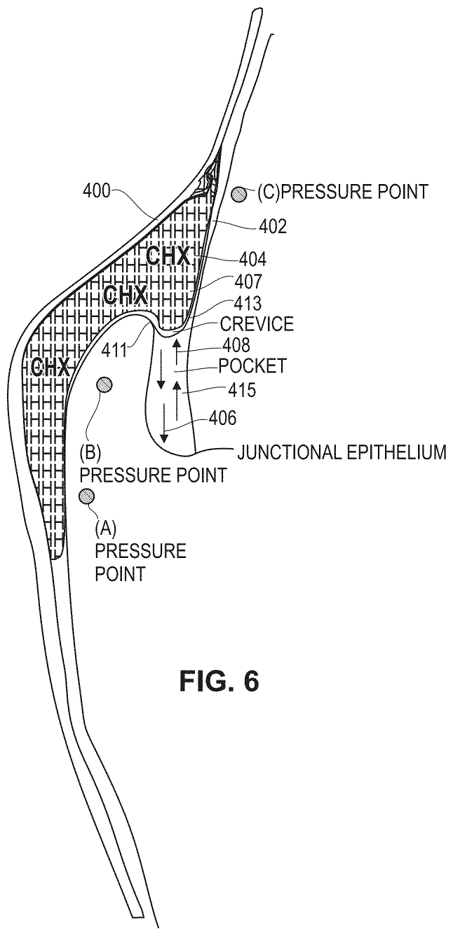

FIG. 6 illustrates an enlarged cross-sectional view of a portion of the oral appliance 400 being worn that is placed adjacent to the teeth and gums using the top down approach to treating periodontal disease. In the embodiment shown, antimicrobial is disposed in a carrier, which is a porous material 402 that is a hydrogel 404 at a discrete region of the oral appliance. The hydrogel is shown in a compressed state 407, where the device is worn and the hydrogel is compressed against, among other things, the gingival crevice or periodontal pocket causing a seal 413 of the entrance of the gingival crevice or periodontal pocket, which prevents oral fluids (e.g., saliva, exudates, foreign liquids, etc.) from entering the crevice or pocket. There is a gap 415 between the junctional epithelium and the entrance 411 of the crevice or pocket, which is now sealed by the hydrogel. This gap allows the hydrogel to release antimicrobial in the gingival crevice or periodontal pocket to treat deep down into the inflamed tissue. The antimicrobial release is shown by the down arrows 406. The hydrogel also absorbs or wicks oral fluids from the crevice or pocket which aides healing, shown by the up arrows 408. The hydrogel is placed in the oral appliance and it is configured to create a seal at the entrance of the periodontal pocket so that there will be a bulge of hydrogel at the entrance to cause such a seal 413.

As the antimicrobial is leached out of the hydrogel, empty hydrogel spaces open up and become available to absorb and remove crevicular/sulcular fluids from the environment. In this way, the hydrogel has dual ability to deliver antimicrobial and wicking action to remove crevicular/sulcular fluids from the environment. This dual action of wicking which then creates a negative crevicular fluid flow, allows the antimicrobials under pressure, shown by pressure points A, B and C, to enter the top portion of the pocket to fill the resultant negative pressure void, thus inserting the antimicrobials further into the pockets. Over sustained daily treatment regimens, the inflammation at the top of the pocket decreases and with decreased inflammation there is decreased swelling and therefore decreased pocket depth.

In some embodiments, the oral appliance has a thickness of from about 0.06 inches to about 0.2 inches. In some embodiments, the oral appliance has a uniform thickness or a non-uniform thickness ranging from about 0.06, 0.07, 0.08, 0.09, 0.10, 0.11, 0.12, 0.13, 0.14, 0.15, 0.16, 0.17, 0.18, 0.19, to about 0.2 inches. The oral appliance can have a uniform or non-uniform thickness of about 0.2 to about 0.5 inches. In some embodiments, the oral appliance comprises a semi-solid construction.

Generally, the goal of periodontal treatment is to decrease inflammation and maintain a reduced inflammatory state. Treating periodontitis with traditional oral hygiene regimens (brushing / flossing) is challenging, as the periodontal pockets are difficult to access. Treating the condition therefore requires, in some cases, a scaling and root planning (SRP) procedure, an invasive in-office mechanical debridement of the periodontal pockets. SRP may be effective, but in practical terms receives patient resistance due to the pain, time, and cost involved. There are no longer-term post-SRP periodontal maintenance therapies that practitioners can prescribe to promote further healing and allow for the proper ongoing management of the chronic condition other than good oral hygiene and antimicrobial rinses. This can be a concerning issue as many patients cannot achieve the level of oral hygiene necessary to maintain periodontal health, and long-term use of antimicrobial rinses is limited due to known side effects. Moreover, it is now understood that oral health is no longer just about killing pathogenic bacteria but rather about maintaining an overall healthy balance of the oral microbiota; rinses that indiscriminately kill bacteria therefore may be deleterious. Given the prevalence of periodontal disease, aging populations, and the increased knowledge of the oral-systemic health connection, there is an urgent need for a non-invasive, easy to administer, clinically effective option to treat and manage periodontal disease. The oral appliance of the current application addresses this need.

Oral Appliance Materials

The oral appliance can be made of any materials that can hold and release the antimicrobial in a carrier or without a carrier. In various embodiments, the material from which the oral appliance can be made from by itself includes swellable polymers, such as for example, hydrogels, gels, polymer brushes or combinations thereof.

In some embodiments, suitable polymers for use to make the oral appliance include, for example, polyacrylates, polyamide-imide, phenolic, nylon, nitrile resins, petroleum resins, fluoropolymers, copolyvidones (copovidones), epoxy, melamine-formaldehyde, diallyl phthalate, acetal, coumarone-indene, acrylics, acrylonitrile-butadiene-styrene, alkyds, cellulosics, polybutylene, polycarbonate, polycaprolactones, polyethylene, polyimides, polyphenylene oxide, polypropylene, polystyrene, polyurethanes, polyvinyl acetates, polyvinyl chloride, poly(vinyl alcohol-co ethylene), styrene acrylonitrile, sulfone polymers, saturated or unsaturated polyesters or combinations thereof.

In some embodiments, the polymer comprises, consists essentially of or consists of an amount from about 5% to about 100% by weight, from about 5%, 10%, 15%, 20%, 25%, 30%, 35%, 40%, 45%, 50%, 55%, 60%, 65%, 70%, 75%, 80%, 85%, 90%, 95% to about 100% by weight, from about 10% to about 15% by weight, from about 15% to about 20% by weight, from about 20% to about 25% by weight, from about 25% to about 30% by weight, from about 30% to about 35% by weight, from about 35% to about 40% by weight, from about 40% to about 45% by weight, from about 45% to about 50% by weight, from about 50% to about 55% by weight, from about 55% to about 60% by weight, from about 60% to about 65% by weight, from about 65% to about 70% by weight, from about 70% to about 75% by weight, from about 75% to about 80% by weight, from about 80% to about 85% by weight, from about 85% to about 90% by weight, from about 90% to about 95% by weight, or from about 95% to about 100% by weight of the oral appliance. In some embodiments, the oral appliance is substantially all polymer from about 80% to about 99.9% by weight. The antimicrobial comprises, consists essentially of or consists of an amount from about 0.001% to about 5% by weight, or about 0.01% to about 50%, from about 0.1% to about 20% by weight, from about 0.5% to about 10%, or from about 1% to about 7% by weight of the oral appliance.

In some embodiments, the antimicrobial (e.g., chlorhexidine) can be disposed in the polymer (e.g., hydrogel) or carrier in an amount of about 0.00001, 0.00005, 0.00010, 0.00015, 0.00020, 0.00025, 0.00030, 0.00035, 0.00040, 0.00045, 0.00050, 0.00055, 0.00060, 0.00065, 0.00070, 0.00075, 0.00080, 0.00085, 0.00090, 0.00095, 0.0010, 0.0015, 0.0020, 0.0025, 0.0030, 0.0035, 0.0040, 0.0045, 0.0050, 0.0055, 0.0060, 0.0065, 0.0070, 0.0075, 0.0080, 0.0085, 0.0090, 0.0095, 0.010, 0.015, 0.020, 0.025, 0.030, 0.035, 0.040, 0.045, 0.050, 0.055, 0.060, 0.065, 0.070, 0.075, 0.080, 0.085, 0.090, 0.095, 0.10, 0.15, 0.20, 0.25, 0.30, 0.35, 0.40, 0.45, 0.50, 0.55, 0.60, 0.65, 0.70, 0.75, 0.80, 0.85, 0.90, 0.95, 1.0, 1.5, 2.0, 2.5, 3.0, 3.5, 4.0, 4.5 to about 5% w/v, w/w and/or v/v based on the total w/v, w/w and/or v/v of the polymer (e.g., hydrogel) or carrier.

In various embodiments, the molecular weight of the polymer can be a wide range of values. The average molecular weight of the polymer can be from about 1000 to about 10,000,000 g/mol; or about 1,000 to about 1,000,000; or about 5,000 to about 500,000; or about 10,000 to about 100,000; or about 20,000 to about 50,000 g/mol.

In some embodiments, when the oral appliance is made from one polymer, the density of the polymer can vary such that the non-porous and porous regions are formed in the oral appliance from a single material.

In some embodiments, when different molecular weight polymers are used, the polymer can be dense and have a higher molecular weight such that the polymer is non-porous. In some embodiments, the polymer can be less dense and have a lower molecular weight such that the polymer is porous. In some embodiments, the oral appliance can be made from multiple polymers, as described above. The multiple polymers can have the same or different densities. The polymers can have an average molecular weight of from about 1000 to about 10,000,000 g/mol; or about 1,000 to about 1,000,000; or about 5,000 to about 500,000; or about 10,000 to about 100,000; or about 20,000 to about 50,000 g/mol.

The polymer can have a modulus of elasticity (Young's modulus) in the range of about 1×10⁻² to about 6×10⁵ dynes/cm², or 2×10⁴ to about 5×10⁵ dynes/cm², or 5×10⁴ to about 5×10⁵ dynes/cm².

The polymer may optionally have a viscosity enhancing agent such as, for example, hydroxypropyl cellulose, hydroxypropyl methylcellulose, hydroxyethyl methylcellulose, carboxymethylcellulose and salts thereof, Carbopol, poly-(hydroxyethylmethacrylate) (pHEMA), poly-(methoxyethylmethacrylate), poly(methoxyethoxyethyl methacrylate), polymethylmethacrylate (PMMA), methylmethacrylate (MMA), gelatin, polyvinyl alcohols, propylene glycol, mPEG, PEG 200, PEG 300, PEG 400, PEG 500, PEG 600, PEG 700, PEG 800, PEG 900, PEG 1000, PEG 1450, PEG 3350, PEG 4500, PEG 8000 or combinations thereof.

In various embodiments, the polymer can comprise a hydrogel that is or is not infused with at least one antimicrobial. Suitable hydrogels for use in the oral appliance, include natural hydrogels, such as for example, gelatin, collagen, silk, elastin, fibrin and polysaccharide-derived polymers like agarose, and chitosan, glucomannan gel, hyaluronic acid, polysaccharides, such as cross-linked carboxyl-containing polysaccharides, or a combination thereof. Synthetic hydrogels include, but are not limited to those formed from polyvinyl alcohol, acrylamides such as polyacrylic acid and poly(acrylonitrile-acrylic acid), polyurethanes, polyethylene glycol (for example, PEG 3350, PEG 4500, PEG 8000), silicone, polyolefins such as polyisobutylene and polyisoprene, copolymers of silicone and polyurethane, neoprene, nitrile, vulcanized rubber, poly(N-vinyl-2-pyrolidone), acrylates such as poly(2-hydroxy ethyl methacrylate) and copolymers of acrylates with N-vinyl pyrolidone, N-vinyl lactams, polyacrylonitrile or combinations thereof.

In some embodiments, cross-linking agents used to make the porous material non-porous include, but are not limited to, glutaraldehyde, formaldehyde, epoxy, compounds, dialdehyde, sodium borate/boric acid, glyoxal, oxidized dextrins, epichlorohydrin, endogen polyamine spermidine, oxidized alginate, zinc, borax, ethylene glycol dimethacrylate (EGDMA), N, N′-methylenebisacrylamide, derivatives of ethylene glycol di(meth)acrylate, derivatives of methylenebisacrylamide, formaldehyde-free crosslinking agent including N-(1-Hydroxy-2,2-dimethoxyethyl)acrylamide, or a combination thereof.

In some embodiments, the oral appliance can be transparent so that a user can see the teeth. The oral appliance may be disposable or sterilizable. In various embodiments, one or more components of the oral appliance is sterilized by radiation in a terminal sterilization step in the final packaging. Terminal sterilization of a product provides greater assurance of sterility than from processes such as an aseptic process, which require individual product components to be sterilized separately and the final package assembled in a sterile environment. Other methods may also be used to sterilize one or more components of the oral appliance, including, but not limited to, E-beam radiation, gamma radiation, gas sterilization, such as, for example, with ethylene oxide or steam sterilization.

In some embodiments, the dimensions of the polymer material (e.g., gel, hydrogel, etc.), among other things, will depend on the target diagnosis site and whether local or systemic collection of the biological material is required as well as the type of biological material collection profile to achieve. In some embodiments, the oral appliance is prepared primarily of polymer material and can be adapted to any size and shape required to receive at least a portion of the teeth and/or soft tissue areas inside the mouth to collect the biological material. For example, the polymer material may, in various embodiments, extend to at least the muco-gingival junction, or at least 2 mm to 5 mm buccally or lingually beyond a gingival margin, or contact all or substantially all of one or more teeth and/or soft tissue areas inside the mouth and adjacent buccal and lingual soft tissue areas.

In various embodiments, the polymer material contacts all or substantially all of one or more teeth and/or soft tissue areas inside the mouth. In various embodiments, the polymer material contacts the soft tissue and teeth at or near a gingival margin or sulcus. In various embodiments, the polymer material has a thickness of from about 0.06 inches to about 0.2 inches, a depth of at least about 1 mm to about 5 mm and a width of from about 1 mm to about 10 mm

### Carrier Material

The oral appliance contains a carrier material which can be located at discrete regions or in a channel on the interior surface, exterior surface or both surfaces of the oral appliance. In many embodiments, the carrier comprises a polymer or a hydrogel. In some embodiments, the polymer includes ethylene glycol dimethacrylate (EGDMA), hydroxypropylcellulose (HPC) or poly(2-hydroxyethyl methacrylate) (pHEMA) or a combination thereof. In some embodiments, the polymer or hydrogel includes from about 80 wt. %, 81%, 82%, 83%, 84%, 85%, 86%, 87%, 88%, 89%, 90%, 91%, 92%, 93%, 94%, 95%, 96%, 97%, 98%, to about 99 wt. % of the carrier. In other embodiments, the polymer or hydrogel includes from about 80% to about 99% weight/weight (w/w), volume/volume (v/v) or weight/volume (w/v) of the carrier.

In some embodiments, the hydrogel is a network of polymer chains that are hydrophilic but water insoluble. Hydrogels are sometimes found as colloidal gels in which water is the dispersion medium. Hydrogels are sometimes superabsorbent (they can contain over 99% water) natural or synthetic polymers.

In various embodiments, the molecular weight of the gel can be varied as desired. The choice of method to vary molecular weight is typically determined by the composition of the gel (e.g., polymer, versus non-polymer). For example, in various embodiments, when the gel comprises one or more polymers, the degree of polymerization can be controlled by varying the amount of polymer initiators (e.g., benzoyl peroxide), organic solvents or activator (e.g., DMPT), crosslinking agents, polymerization agent, incorporation of chain transfer or chain capping agents and/or reaction time.

In various embodiments, when the hydrogel is polymerized by UV curing, a photoinitiator can be used. Nonlimiting example of commercially available photoinitiators include diethoxyacetophenone (DEAP), dimethoxyphenylacetophenone (Irgacure 651), benzoylcyclohexanol (Irgacure 184), or hydroxydimethylacetophenone (Darocure 1173). In some embodiments, the amount of photoinitiator can vary from about 0.015, 0.020, 0.025, 0.030, 0.035, 0.040, 0.045, 0.050, 0.055, 0.060, 0.065, 0.070, to about 0.075 mL.

Suitable gel polymers may be soluble in an organic solvent. The solubility of a polymer in a solvent varies depending on the crystallinity, hydrophobicity, hydrogen-bonding and molecular weight of the polymer. Lower molecular weight polymers will normally dissolve more readily in an organic solvent than high-molecular weight polymers. A polymeric gel that includes a high molecular weight polymer tends to coagulate or solidify more quickly than a polymeric composition that includes a low-molecular weight polymer. Polymeric gel formulations that include high molecular weight polymers also tend to have a higher solution viscosity than a polymeric gel that includes low-molecular weight polymers. In various embodiments, the molecular weight of the polymer can be a wide range of values. The average molecular weight of the polymer can be from about 1000 to about 10,000,000; or about 1,000 to about 1,000,000; or about 5,000 to about 500,000; or about 10,000 to about 100,000; or about 20,000 to 50,000 g/mol; or about 300 kDa to about 1,000 kDa.

In various embodiments, the gel has an inherent viscosity (abbreviated as “I.V.” and units are in deciliters/gram), which is a measure of the gel's molecular weight and degradation time (e.g., a gel with a high inherent viscosity has a higher molecular weight and may have a longer degradation time). Typically, when the polymers have similar components but different molecular weights, a gel with a high molecular weight provides a stronger matrix and the matrix takes more time to degrade. In contrast, a gel with a low molecular weight degrades more quickly and provides a softer matrix. In various embodiments, the gel has a molecular weight, as shown by the inherent viscosity, from about 0.10 dL/g to about 1.2 dL/g or from about 0.10 dL/g to about 0.40 dL/g. Other IV ranges include but are not limited to about 0.05 to about 0.15 dL/g, about 0.10 to about 0.20 dL/g, about 0.15 to about 0.25 dL/g, about 0.20 to about 0.30 dL/g, about 0.25 to about 0.35 dL/g, about 0.30 to about 0.35 dL/g, about 0.35 to about 0.45 dL/g, about 0.40 to about 0.45 dL/g, about 0.45 to about 0.55 dL/g, about 0.50 to about 0.70 dL/g, about 0.60 to about 0.80 dL/g, about 0.70 to about 0.90 dL/g, about 0.80 to about 1.00 dL/g, about 0.90 to about 1.10 dL/g, about 1.0 to about 1.2 dL/g, about 1.1 to about 1.3 dL/g, about 1.2 to about 1.4 dL/g, about 1.3 to about 1.5 dL/g, about 1.4 to about 1.6 dL/g, about 1.5 to about 1.7 dL/g, about 1.6 to about 1.8 dL/g, about 1.7 to about 1.9 dL/g, and about 1.8 to about 2.1 dL/g.

In some embodiments, when the polymer materials have different chemistries (e.g., high MW DLG 5050 and low MW DL), the high MW polymer may degrade faster than the low MW polymer.

In various embodiments, the gel can have a viscosity of about 300 to about 5,000 centipoise (cp). In other embodiments, the gel can have a viscosity of from about 5 to about 300 cps, from about 10 cps to about 50 cps, or from about 15 cps to about 75 cps at room temperature. The gel may optionally have a viscosity enhancing agent such as, for example, hydroxypropyl cellulose, hydroxypropyl methylcellulose, hydroxyethyl methylcellulose, carboxymethylcellulose and salts thereof, Carbopol, poly-(hydroxyethylmethacrylate), poly-(methoxyethylmethacrylate), poly(methoxyethoxyethyl methacrylate), polymethylmethacrylate (PMMA), methylmethacrylate (MMA), gelatin, polyvinyl alcohols, propylene glycol, mPEG, PEG 200, PEG 300, PEG 400, PEG 500, PEG 600, PEG 700, PEG 800, PEG 900, PEG 1000, PEG 1450, PEG 3350, PEG 4500, PEG 8000 or combinations thereof.

In various embodiments, the gel is a hydrogel made of high molecular weight biocompatible elastomeric polymers of synthetic or natural origin. In other embodiments, the hydrogel material can hold collected biological materials when the hydrogel material is hypo-saturated, saturated, or supersaturated. There are many advantages resulting from using hydrogel as the carrier for antimicrobials used in the oral appliances described herein. Generally, hydrogel materials provide an effective contact medium for gum compression and for collecting biological materials for diagnosis. The above can hold the sample (e.g., saliva, blood, cells, various oral fluids, inflammatory factors, other biologic markers, etc.) when the oral appliance is removed and then the oral appliance can be sent to the lab for testing. Sending out the entire oral appliance to the lab can prevent cross contamination by the patient's hands contaminating the sample collected by the hydrogel. However, in another way, in some embodiments, only the hydrogel carrier can be removed and then sent out to the lab for testing.

In various embodiments, useful materials for preparing the carrier for the antimicrobial used in the oral appliances described in this disclosure comprise reactive segmented block copolymers containing hydrophilic domain(s) and showing good surface properties when the block copolymer is covalently bound to substrates containing complimentary functionality. The hydrophilic domain(s) will comprise at least one hydrophilic monomer, such as, HEMA, glyceryl methacrylate, methacrylic acid (“MAA”), acrylic acid (“AA”), methacrylamide, acrylamide, N,N′-dimethylmethacrylamide, or N,N′-dimethylacrylamide; copolymers thereof; hydrophilic prepolymers, such as ethylenically unsaturated poly(alkylene oxide)s, cyclic lactams such as N-vinyl-2-pyrrolidone (“NVP”), or derivatives thereof. Still further examples are the hydrophilic vinyl carbonate or vinyl carbamate monomers. Hydrophilic monomers can be nonionic monomers, such as 2-hydroxyethyl methacrylate (“HEMA”), 2-hydroxyethyl acrylate (“HEA”), 2-(2-ethoxyethoxy)ethyl(methacrylate), glyceryl(meth)acrylate, poly(ethylene glycol(methacrylate), tetrahydrofurfuryl(methacrylate), (methacrylamide), N,N′-dimethylmethacrylamide, N,N′-dimethylacrylamide (“DMA”), N-vinyl-2-pyrrolidone (or other N-vinyl lactams), N-vinyl acetamide, and combinations thereof. Still further examples of hydrophilic monomers are the vinyl carbonate and vinyl carbamate monomers disclosed in U.S. Pat. No. 5,070,215, and the hydrophilic oxazolone monomers disclosed in U.S. Pat. No. 4,910,277. The contents of these patents are incorporated herein by reference. The hydrophilic monomer also can be an anionic monomer, such as 2-methacryloyloxyethylsulfonate salts. Substituted anionic hydrophilic monomers, such as from acrylic and methacrylic acid, can also be utilized wherein the substituted group can be removed by a facile chemical process.

In various embodiments, monomers and/or copolymers useful in preparing the hydrogel carriers for the oral appliances described in this application include without limitation 2-hydroxyethyl methacrylate (HEMA), polyvinyl alcohol (PVA), ethylene glycol dimethacrylate (EGDMA) and/or poly-2-hydroxyethyl methacrylate (pHEMA) or combinations thereof. Polymers useful in preparing the hydrogel carriers for the oral appliances described in this application include without limitation poly-2-hydroxyethyl methacrylate (pHEMA), poly(ethylene glycol) diacrylate (PEGDA) or hydroxypropyl cellulose (HPC) or combinations thereof. In some aspects, pHEMA can have various molecular weights, for example, 300,000 Da or 1,000,000 Da. In various aspects, the amount of pHEMA useful for preparing the hydrogels described in this disclosure varies from about 15% to about 50 w/w, v/v or w/v and the amount of EGDMA varies from about 0.001% to about 0.005% w/w, v/v or w/v based on a total weight or a total volume of the hydrogel.

### Antimicrobials

The oral appliance contains one or more antimicrobials disposed in or on the oral appliance. The antimicrobial can be disposed in a carrier as discussed above. The carrier can be a polymer. In various embodiments, some areas of the polymer material of the oral appliance do not contain one or more antimicrobials, and the polymer material may function to hold or lock a portion of the polymer material in place so that other portions of the polymer material can contact the appropriate target site. Thus, in some embodiments, the polymer material may contain one or more antimicrobials disposed in or on it throughout the whole polymer material of the oral appliance. In other embodiments, one or more portions of the oral appliance do not contain any antimicrobial disposed in or on it. In many embodiments, the antimicrobial is disposed in the carrier at discrete regions of the interior surface, the exterior surface or both interior and exterior surface for delivering the antimicrobial to the oral cavity. In various embodiments, the antimicrobial is present in the carrier in an amount of about 0.01% w/w, v/v or w/v to about 20% w/w, v/v or w/v based on a total weight or a total volume of the carrier. In other embodiments, the antimicrobial is present in the carrier in an amount of about 1.0% w/w, v/v or w/v to about 20% w/w, v/v or w/v based on a total weight or a total volume of the carrier.

In some embodiments, the antimicrobial (e.g., chlorhexidine) can be disposed in the polymer (e.g., hydrogel) or carrier in an amount of about 0.00001, 0.00005, 0.00010, 0.00015, 0.00020, 0.00025, 0.00030, 0.00035, 0.00040, 0.00045, 0.00050, 0.00055, 0.00060, 0.00065, 0.00070, 0.00075, 0.00080, 0.00085, 0.00090, 0.00095, 0.0010, 0.0015, 0.0020, 0.0025, 0.0030, 0.0035, 0.0040, 0.0045, 0.0050, 0.0055, 0.0060, 0.0065, 0.0070, 0.0075, 0.0080, 0.0085, 0.0090, 0.0095, 0.010, 0.015, 0.020, 0.025, 0.030, 0.035, 0.040, 0.045, 0.050, 0.055, 0.060, 0.065, 0.070, 0.075, 0.080, 0.085, 0.090, 0.095, 0.10, 0.15, 0.20, 0.25, 0.30, 0.35, 0.40, 0.45, 0.50, 0.55, 0.60, 0.65, 0.70, 0.75, 0.80, 0.85, 0.90, 0.95, 1.0, 1.5, 2.0, 2.5, 3.0, 3.5, 4.0, 4.5 to about 5% w/v, w/w and/or v/v based on the total w/v, w/w and/or v/v of the polymer (e.g., hydrogel) or carrier.

The antimicrobial may be in powder, liquid, solid, solution, or suspension (e.g., hydrogel) form and disposed on or impregnated in the oral appliance. This may occur during manufacture of the oral appliance or it may occur after the oral appliance is made. For example, on the core polymer material of the oral appliance, the antimicrobial may be layered by solution or suspension layering or powder layering techniques. In solution or suspension layering, the antimicrobial and any inactive ingredients (excipients, binders, etc.) are suspended or dissolved in water or an organic solvent. The resulting liquid is sprayed onto the outside of the oral appliance to make the polymer material have the desired potency. Solution or suspension layering may be conducted using a wide variety of process techniques, for example, by fluidized bed, Wurster bottom spray techniques, or the like. When the desired potency has been achieved, the polymer material is dried to the desired residual moisture content. Powdered layering involves the application of a dry powder to the oral appliance. The powder may contain the antimicrobial, or may include excipients such as a binder, flow aid, inert filler, and the like. In the powder layering technique, a pharmaceutically acceptable liquid, which may be water, organic solvent, with or without a binder and/or excipient, is applied to the oral appliance while applying the dry powder until the desired potency is achieved. When the desired potency has been achieved, the oral appliance may be dried to the desired moisture content.

In various embodiments, the antimicrobial is in liquid form and is capable of diffusing through and within the oral appliance comprising a polymer material. In various embodiments, the liquid antimicrobial may flow or diffuse from one portion of the oral appliance to another portion. In some embodiments, the liquid antimicrobial may not flow or diffuse within the oral appliance. In some embodiments, the liquid antimicrobial is confined within the regions of the oral appliance corresponding to the treatment area. The liquid antimicrobial is not capable of flowing or diffusing into the non-porous regions of the oral appliance.

Examples of antimicrobial agents to treat infection include without limitation, antiseptic agents, antibacterial agents; quinolones and in particular fluoroquinolones (e.g., norfloxacin, ciprofloxacin, lomefloxacin, ofloxacin, etc.), aminoglycosides (e.g., gentamicin, tobramycin, etc.), glycopeptides (e.g., vancomycin, etc.), lincosamides (e.g., clindamycin), cephalosporins (e.g., first, second, third generation) and related beta-lactams, macrolides (e.g., azithromycin, erythromycin, etc.), nitroimidazoles (e.g., metronidazole), penicillins, polymyxins, tetracyclines, or combinations thereof.

Some exemplary antimicrobial agents include, by way of illustration and not limitation, acedapsone; acetosulfone sodium; alamecin; alexidine; amdinocillin; amdinocillin pivoxil; amicycline; amifloxacin; amifloxacin mesylate; amikacin; amikacin sulfate; aminosalicylic acid; aminosalicylate sodium; amoxicillin; amphomycin; ampicillin; ampicillin sodium; apalcillin sodium; apramycin; aspartocin; astromicin sulfate; avilamycin; avoparcin; azithromycin; azlocillin; azlocillin sodium; bacampicillin hydrochloride; bacitracin; bacitracin methylene disalicylate; bacitracin zinc; bambermycins; benzoylpas calcium; berythromycin; betamicin sulfate; biapenem; biniramycin; biphenamine hydrochloride; bispyrithione magsulfex; butikacin; butirosin sulfate; capreomycin sulfate; carbadox; carbenicillin disodium; carbenicillin indanyl sodium; carbenicillin phenyl sodium; carbenicillin potassium; carumonam sodium; cefaclor; cefadroxil; cefamandole; cefamandole nafate; cefamandole sodium; cefaparole; cefatrizine; cefazaflur sodium; cefazolin; cefazolin sodium; cefbuperazone; cefdinir; cefepime; cefepime hydrochloride; cefetecol; cefixime; cefmenoxime hydrochloride; cefmetazole; cefmetazole sodium; cefonicid monosodium; cefonicid sodium; cefoperazone sodium; ceforanide; cefotaxime sodium; cefotetan; cefotetan disodium; cefotiam hydrochloride; cefoxitin; cefoxitin sodium; cefpimizole; cefpimizole sodium; cefpiramide; cefpiramide sodium; cefpirome sulfate; cefpodoxime proxetil; cefprozil; cefroxadine; cefsulodin sodium; ceftazidime; ceftibuten; ceftizoxime sodium; ceftriaxone sodium; cefuroxime; cefuroxime axetil; cefuroxime pivoxetil; cefuroxime sodium; cephacetrile sodium; cephalexin; cephalexin hydrochloride; cephaloglycin; cephaloridine; cephalothin sodium; cephapirin sodium; cephradine; cetocycline hydrochloride; cetophenicol; chloramphenicol; chloramphenicol palmitate; chloramphenicol pantothenate complex; chloramphenicol sodium succinate; chlorhexidine phosphanilate; chloroxylenol; chlortetracycline bisulfate; chlortetracycline hydrochloride; cinoxacin; ciprofloxacin; ciprofloxacin hydrochloride; cirolemycin; clarithromycin; clinafloxacin hydrochloride; clindamycin; clindamycin hydrochloride; clindamycin palmitate hydrochloride; clindamycin phosphate; clofazimine; cloxacillin benzathine; cloxacillin sodium; chlorhexidine, cloxyquin; colistimethate sodium; colistin sulfate; coumermycin; coumermycin sodium; cyclacillin; cycloserine; dalfopristin; dapsone; daptomycin; demeclocycline; demeclocycline hydrochloride; demecycline; denofungin; diaveridine; dicloxacillin; dicloxacillin sodium; dihydrostreptomycin sulfate; dipyrithione; dirithromycin; doxycycline; doxycycline calcium; doxycycline fosfatex; doxycycline hyclate; droxacin sodium; enoxacin; epicillin; epitetracycline hydrochloride; erythromycin; erythromycin acistrate; erythromycin estolate; erythromycin ethylsuccinate; erythromycin gluceptate; erythromycin lactobionate; erythromycin propionate; erythromycin stearate; ethambutol hydrochloride; ethionamide; fleroxacin; floxacillin; fludalanine; flumequine; fosfomycin; fosfomycin tromethamine; fumoxicillin; furazolium chloride; furazolium tartrate; fusidate sodium; fusidic acid; ganciclovir and ganciclovir sodium; gentamicin sulfate; gloximonam; gramicidin; haloprogin; hetacillin; hetacillin potassium; hexedine; ibafloxacin; imipenem; isoconazole; isepamicin; isoniazid; josamycin; kanamycin sulfate; kitasamycin; levofuraltadone; levopropylcillin potassium; lexithromycin; lincomycin; lincomycin hydrochloride; lomefloxacin; lomefloxacin hydrochloride; lomefloxacin mesylate; loracarbef; mafenide; meclocycline; meclocycline sulfosalicylate; megalomicin potassium phosphate; mequidox; meropenem; methacycline; methacycline hydrochloride; methenamine; methenamine hippurate; methenamine mandelate; methicillin sodium; metioprim; metronidazole hydrochloride; metronidazole phosphate; mezlocillin; mezlocillin sodium; minocycline; minocycline hydrochloride; mirincamycin hydrochloride; monensin; monensin sodiumr; nafcillin sodium; nalidixate sodium; nalidixic acid; natainycin; nebramycin; neomycin palmitate; neomycin sulfate; neomycin undecylenate; netilmicin sulfate; neutramycin; nifuiradene; nifuraldezone; nifuratel; nifuratrone; nifurdazil; nifurimide; nifiupirinol; nifurquinazol; nifurthiazole; nitrocycline; nitrofurantoin; nitromide; norfloxacin; novobiocin sodium; ofloxacin; onnetoprim; oxacillin and oxacillin sodium; oximonam; oximonam sodium; oxolinic acid; oxytetracycline; oxytetracycline calcium; oxytetracycline hydrochloride; paldimycin; parachlorophenol; paulomycin; pefloxacin; pefloxacin mesylate; penamecillin; penicillins such as penicillin g benzathine, penicillin g potassium, penicillin g procaine, penicillin g sodium, penicillin v, penicillin v benzathine, penicillin v hydrabamine, and penicillin v potassium; pentizidone sodium; phenyl aminosalicylate; piperacillin sodium; pirbenicillin sodium; piridicillin sodium; pirlimycin hydrochloride; pivampicillin hydrochloride; pivampicillin pamoate; pivampicillin probenate; polymyxin b sulfate; porfiromycin; propikacin; pyrazinamide; pyrithione zinc; quindecamine acetate; quinupristin; racephenicol; ramoplanin; ranimycin; relomycin; repromicin; rifabutin; rifametane; rifamexil; rifamide; rifampin; rifapentine; rifaximin; rolitetracycline; rolitetracycline nitrate; rosaramicin; rosaramicin butyrate; rosaramicin propionate; rosaramicin sodium phosphate; rosaramicin stearate; rosoxacin; roxarsone; roxithromycin; sancycline; sanfetrinem sodium; sarmoxicillin; sarpicillin; scopafungin; sisomicin; sisomicin sulfate; sparfloxacin; spectinomycin hydrochloride; spiramycin; stallimycin hydrochloride; steffimycin; streptomycin sulfate; streptonicozid; sulfabenz; sulfabenzamide; sulfacetamide; sulfacetamide sodium; sulfacytine; sulfadiazine; sulfadiazine sodium; sulfadoxine; sulfalene; sulfamerazine; sulfameter; sulfamethazine; sulfamethizole; sulfamethoxazole; sulfamonomethoxine; sulfamoxole; sulfanilate zinc; sulfanitran; sulfasalazine; sulfasomizole; sulfathiazole; sulfazamet; sulfisoxazole; sulfisoxazole acetyl; sulfisboxazole diolamine; sulfomyxin; sulopenem; sultamricillin; suncillin sodium; talampicillin hydrochloride; teicoplanin; temafloxacin hydrochloride; temocillin; tetracycline; tetracycline hydrochloride; tetracycline phosphate complex; tetroxoprim; thiamphenicol; thiphencillin potassium; ticarcillin cresyl sodium; ticarcillin disodium; ticarcillin monosodium; ticlatone; tiodonium chloride; tobramycin; tobramycin sulfate; tosufloxacin; trimethoprim; trimethoprim sulfate; trisulfapyrimidines; troleandomycin; trospectomycin sulfate; tyrothricin; vancomycin; vancomycin hydrochloride; virginiamycin; zorbamycin; or combinations thereof.

Antiviral agents can include, but are not limited to, vidarabine, acyclovir, famciclovir, valacyclovir, gancyclovir, valganciclovir, nucleoside-analog reverse transcriptase inhibitors (such as AZT (zidovudine), ddI (didanosine), ddC (zalcitabine), d4T (stavudine), and 3TC (lamivudine)), nevirapine, delavirdine, protease inhibitors (such as, saquinavir, ritonavir, indinavir, and nelfinavir), ribavirin, amantadine, rimantadine, neuraminidase inhibitors (such as zanamivir and oseltamivir), pleconaril, cidofovir, foscarnet, and/or interferons.

Antifungal agents that can be used in the oral appliance include, but are not limited to, nystatin, clotrimazole, griseofulvin, ketoconazole, itraconazole, fluconazole, terbinafine, or a combination thereof.

Antimicrobials include antiseptics. Suitable antiseptics that can be in the carrier (e.g., polymer) include chlorhexidine, chlorhexidine gluconate, chlorhexidine digluconate, hexetidine, hydrogen peroxide, sodium hypochlorite, cetylpyridinium chloride, triclosan, methyl salicylate, povidone-iodine, alcohol, boric acid, iodine, hexachlorophene, or a combination thereof. Astrigents, for example, zinc chloride are also useful excipients for the oral appliances described in this disclosure.

Oral appliances are provided that can deliver antimicrobials to at least a portion of the teeth and/or soft tissues inside the oral cavity in a three-dimensional way. One advantage of the oral appliance is that it is custom made to fit only one patient. As used herein a “custom fit” oral appliance refers to an oral appliance prepared to correspond to at least a portion of the teeth or all of the teeth and soft tissues of a specific patient. Typically, the custom fit appliance is prepared by a dental care professional (e.g., dentist, oral surgeon, medical doctor, other health care professional, manufacturer, etc.). The custom fit oral appliance can be made from an impression mold or using an analog or digital image capturing device. The oral appliance provided by this disclosure is not a boil-and-bite prefabricated device or a stock oral appliance, which can be manipulated by the consumer himself/herself with fingers to shape it against the teeth and gums, but which cannot be shaped to properly align the antimicrobials with the proper geographic anatomy.

In some embodiments, the oral appliance can contain antimicrobials separately in a cargo area or sponge or placed as a liquid in the oral appliance. The oral appliances disclosed herein are custom fit, disposable, and manufactured in one continuous step, pre-loaded with antimicrobial in or on at least a portion of the interior and/or exterior surfaces of the appliance and can deliver antimicrobials. In some embodiments, the oral appliance can be transparent. Still another advantage of the oral appliance is that, in various embodiments, it can be easily manufactured and is comfortable for the patient to use. Other advantages of the oral appliances provided by this disclosure include greater efficacy over conventional oral therapies based on two dimensional systems, user convenience, enhanced patient compliance, less dilution of antimicrobial and enhanced applied pressure to gums.

In one embodiment, there is an oral appliance for delivering an antimicrobial to at least a portion of teeth and/or soft tissue areas inside an oral cavity, the oral appliance comprising an interior surface having an antimicrobial disposed in or on at least a portion of and/or all of the interior surface of the oral appliance, the interior surface being formed to fit contours of at least the portion of the teeth and/or soft tissue, including inflamed soft tissue areas inside the oral cavity and being configured for holding the antimicrobial in contact with at least the portion of the teeth and/or soft tissue areas inside the oral cavity to deliver the antimicrobial thereto.

The soft tissue of the inside of the mouth includes, but is not limited to, any soft tissue adjacent or between the teeth including, but not limited to, the papilla, tissue of the upper and lower dental arches, marginal gingiva, gingival sulcus, inter-dental gingiva, gingival gum structure on lingual and buccal surfaces up to and including the muco-gingival junction and/or the upper palate and/or the floor of the mouth.

In various embodiments, the soft tissue area inside the oral cavity includes the muco-buccal folds, hard and soft palates, lining mucosa, the tongue (one or all surfaces of the tongue) and/or attached gingival tissue, all of which may occasionally become inflamed as caused by any number of conditions. In various embodiments, the oral appliance receives one or more teeth including one or more molars, premolars, incisors, cuspids, tooth implant, or combination or portions thereof. In other embodiments, the antimicrobial contained in the oral appliance can be disposed anywhere in or on the interior or exterior surface of the oral appliance adjacent to the gum and/or other soft tissue areas of the oral cavity including the mesial, distal, buccal, labial, lingual, palatal and occlusal surfaces of one or more teeth.

In various embodiments, the oral appliance may contain more than one antimicrobial. However, in another embodiment, combination therapy will involve use of a single, safe and effective amount of the antimicrobial. For example, the method may further comprise subsequently administering one or more additional oral appliances, each containing an antimicrobial that is different from the antimicrobial contained in the earlier oral appliance. In this way, a series of customized treatment regimens can be provided to the patient. This provides for a “mix and match” antimicrobial regimen with dose adjustment capability and provides the added advantage of allowing the health professional complete control to administer only those antimicrobials at the desired strength believed to be appropriate for the disease or condition being treated to a particular individual.

In some embodiments, one or more oral appliances can be administered to a patient to treat inflammation and/or pain or other conditions associated with inflammation.

The amount of antimicrobial contained within the oral appliance, will vary widely depending on the effective dosage required and rate of release from the polymer material and the length of the desired delivery interval. The dosage administered to the patient can be single or multiple doses and will vary depending upon a variety of factors, including the agent's pharmacokinetic properties, patient conditions and characteristics (sex, age, body weight, health, size, etc.), extent of symptoms, concurrent treatments, frequency of treatment and the effect desired. These factors can readily be determined by those of ordinary skill in the art.

In various embodiments, the polymer material of the oral appliance is designed to release the antimicrobial as a bolus dose of the antimicrobial, a single dose of the antimicrobial, or multiple doses of the antimicrobial all preloaded with a specific dosage at the manufacturing facility.

In some embodiments, the amount of antimicrobial, disposed in the carrier can be in an amount of from about 0.01% to about 20%, from about 0.1% to about 20%, from about 1% to about 20%, from about 1% to about 10% or from about 1% to 5% w/w, v/v or w/v based on the weight of the oral appliance. The amount of antimicrobial disposed in the carrier can be in an amount of from about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2, 3, 4, 5, 6, 7, 8, 9, 10, 11, 12, 13, 14, 15, 16, 17, 18, 19, to about 20% based on the weight of the oral appliance. For example, a first oral appliance can be made specific or custom fit for a patient. The oral appliance can have porous and non-porous regions, the antimicrobial can be in the porous and/or non-porous regions, for example, from about 0.01% to about 5% w/w, v/v or w/v chlorhexidine based on the total weight of the oral appliance or polymer carrier. A second oral appliance can be made for that patient with the same or a lesser amount of antimicrobial, for example 2.5% or 0.01% chlorhexidine based on the total weight of the oral appliance or polymer carrier.

The amount of antimicrobial contained within the oral appliance, will vary widely depending on the effective dosage required and rate of release from the polymer material, the length of the desired delivery interval and the surface area to be covered by the medicament/hydrogel. The dosage administered to the patient can be single or multiple doses and will vary depending upon a variety of factors, including the agent's pharmacokinetic properties, patient conditions and characteristics (sex, age, body weight, health, size, etc.), extent of symptoms, concurrent treatments, frequency of treatment and the effect desired. These factors can readily be determined by those of ordinary skill in the art.

In various embodiments, the polymer material of the oral appliance is designed to release the antimicrobial as a bolus dose of the antimicrobial, a single dose of the antimicrobial, or multiple doses of the antimicrobial all preloaded with a specific dosage at the manufacturing facility.

In some embodiments, the antimicrobial described herein is in the carrier of the oral appliance in an amount of from about 0.01, 0.02, 0.03, 0.04, 0.05, 0.06, 0.07, 0.08, 0.09, 1, 2, 3, 4, 5, 6, 7, 8, 9, 10, 11, 12, 13, 14, 15, 16, 17, 18, 19, to about 20% by weight of the carrier.

In some embodiments, the antimicrobial (e.g., chlorhexidine) can be disposed in the polymer (e.g., hydrogel) or carrier in an amount of about 0.00001, 0.00005, 0.00010, 0.00015, 0.00020, 0.00025, 0.00030, 0.00035, 0.00040, 0.00045, 0.00050, 0.00055, 0.00060, 0.00065, 0.00070, 0.00075, 0.00080, 0.00085, 0.00090, 0.00095, 0.0010, 0.0015, 0.0020, 0.0025, 0.0030, 0.0035, 0.0040, 0.0045, 0.0050, 0.0055, 0.0060, 0.0065, 0.0070, 0.0075, 0.0080, 0.0085, 0.0090, 0.0095, 0.010, 0.015, 0.020, 0.025, 0.030, 0.035, 0.040, 0.045, 0.050, 0.055, 0.060, 0.065, 0.070, 0.075, 0.080, 0.085, 0.090, 0.095, 0.10, 0.15, 0.20, 0.25, 0.30, 0.35, 0.40, 0.45, 0.50, 0.55, 0.60, 0.65, 0.70, 0.75, 0.80, 0.85, 0.90, 0.95, 1.0, 1.5, 2.0, 2.5, 3.0, 3.5, 4.0, 4.5 to about 5% w/v, w/w and/or v/v based on the total w/v, w/w and/or v/v of the polymer (e.g., hydrogel) or carrier.

In some embodiments, the antimicrobial can be disposed anywhere in or on the interior or exterior surface of the oral appliance adjacent to the gum and/or other soft tissue areas of the oral cavity including the front, back, occlusal surfaces of one or more teeth. Some portions of teeth that do not require the antimicrobial are sealed with the non-porous material which can be a coating, cross-linked with a porosity reducing agent or comprising non-porous material such that the antimicrobial cannot penetrate said portions. In some embodiments, the antimicrobial may be disposed in or may enter the non-porous region. However, the antimicrobial disposed in the non-porous region will not release the antimicrobial or will release the antimicrobial at a reduced rate.

In some embodiments, the antimicrobial may enter the non-porous regions, but the antimicrobial will be released more slowly from these regions. For example, the antimicrobial can be disposed at discrete non-porous regions adjacent to the treatment area or uniformly disposed throughout the device. In this example, the antimicrobial will not be released to other regions that do not correspond with the treatment area.

In some embodiments, the antimicrobial may flow into the non-porous regions but the antimicrobial in the non-porous regions will be released at a slower rate than that of the porous regions. As the interior and/or exterior surface of the oral appliance contacts the oral cavity, the antimicrobial is released from the polymer such that all or parts of the oral appliance will degrade over time by the action of enzymes, by hydrolytic action and/or by other similar mechanisms in the oral cavity. In various embodiments, the degradation can occur either at the surface of the oral appliance at discrete positions (heterogeneous or surface erosion) or uniformly throughout the oral appliance (homogeneous or bulk erosion). In some embodiments, all or discrete portions of the interior surface will degrade, particularly those regions containing the hydrogel having the antimicrobial, and release antimicrobial at or near the target site in the oral cavity. The oral appliance will cover at least a portion of the teeth and or gums, by applying the device over axis to cover the area of the teeth and or gums, and the oral appliance will be adjacent to the gingival sulcus or other soft tissue or hard tissue areas, which will allow the antimicrobial, if desired, to be released from the polymer to these areas.

Chlorhexidine is an antimicrobial used as an antiseptic and disinfectant effective against a wide variety of gram-positive and gram-negative bacteria, fungi, yeast and select viruses. Chlorhexidine has been used since 1959 and is widely available throughout the world. Chemically, chlorhexidine is a strong base and is most stable in its salt forms. Chlorhexidine gluconate (1,1′-hexamethylene bis [5-(p-chlorophenyl biguanide]di-D-gluconate), also known as chlorhexidine digluconate, is a salt formed from chlorhexidine and gluconic acid.

Chlorhexidine salts are adsorbed onto the cell walls of microorganisms, resulting in disruption of the cell wall integrity and leakage of intracellular contents. At low concentrations, chlorhexidine is a bacteriostatic agent, and at higher concentrations it becomes bactericidal. A primary benefit of chlorhexidine is its ability to kill bacteria on contact and remain non-toxic to mammalian cells.

Chlorhexidine salts are cationic, which facilitates their adsorption onto the surfaces of the oral mucosa, teeth and plaque, all of which have a net negative charge. The adsorbed chlorhexidine is gradually released from these tissues by diffusion. Thus, chlorhexidine has a substantial residual effect in that it retards microbial growth in the mouth for prolonged periods after application, allowing for either interval use or for daily application.

Chlorhexidine has been marketed for use in the oral cavity in many forms, including mouthwashes (usually 0.1-0.2%), 2% topical oral drops, lozenges, implantable chips, etc. In many countries, these preparations are sold over the counter. In the United States, chlorhexidine gluconate is available via prescription and over the counter (OTC).

In the U.S., chlorhexidine for dental use is limited to prescription status and is available as an oral rinse and as a 2.5 mg chip (PerioChip®-Astra). The chip contains 2.5 mg of chlorhexidine gluconate in a glycerin and gelatin matrix and is indicated as an adjunct in scaling and root planning procedures for the reduction of a single pocket depth for each chip placed in patients with adult periodontitis (Drug Facts and Comparisons, 1999). Chlorhexidine gluconate rinse is available in the U.S. as a 0.12% solution (1.2 mg/m1) for the treatment of gingivitis. This commercial rinse is usually flavored with anise or mint and contains 11.6% (23 proof) alcohol by weight.

Any suitable source of chlorhexidine can be used in the compositions and methods of this disclosure. Suitable chlorhexidine starting materials include chlorhexidine salts, as they have enhanced stability over the parent chlorhexidine. In various embodiments described in this disclosure, chlorhexidine gluconate (also known as chlorhexidine digluconate), is a useful salt due to its high-water solubility. Other useful compounds include chlorhexidine diacetate and chlorhexidine dihydrochloride.

In various embodiments, chlorhexidine useful for the oral appliances of this disclosure comprises from about 0.01, 0.02, 0.03, 0.04, 0.05, 0.06, 0.07, 0.08, 0.09, 1, 2, 3, 4, 5, 6, 7, 8, 9, 10, 11, 12, 13, 14, 15, 16, 17, 18, 19, to about 20% by weight of the carrier of the oral appliance. In many embodiments, the carrier is hydrogel.

In various embodiments, the oral appliance for delivering an antimicrobial to an oral cavity includes other excipients in the carrier or the antimicrobial composition described in this disclosure. Generally, “excipient” refers to an inert or inactive substance used in the production of pharmaceutical products. Useful excipients for the antimicrobial compositions of this disclosure include without limitation emollients and surfactants. These agents are chosen that do not interfere with the antimicrobial activity and do not rot the teeth and/or gums. In some aspects, the antimicrobial composition can include an alcohol, and, in other aspects, the composition can be alcohol free.

Antioxidants such as vitamin E or coenzyme Q or a colorant in non-toxic concentration are also useful excipients that can be added to the carrier or antimicrobial composition of this disclosure.

### Methods of Making the Oral Appliance

The oral appliance is custom made to fit a specific patient. The custom-made oral appliance may be prepared by a dental care professional including, but not limited to a dentist, oral surgeon, medical doctor, technician or manufacturer. The oral appliance can be made from an impression mold, or by using an analog or digital image capturing device. The oral appliance disclosed herein is not a boil and bite prefabricated device or a stock tray which can be manipulated by the consumer himself/herself with fingers to shape it against the teeth and gums. The oral appliance disclosed herein is custom fit, disposable, and monolithic and is pre-loaded with antimicrobial into or onto at least a portion of the interior and/or exterior surfaces of the appliance and can deliver antimicrobial agents as more particularly described in U.S. Patent Application No. 15/895,554 to Peter J. Zegarelli, filed on Feb. 13, 2018. The entire disclosure of this application is incorporated herein by reference into the present application.

In some embodiments, the dental practitioner identifies the region that requires the antimicrobial and the region that does not require the antimicrobial. The region that requires the antimicrobial is made with porous material infused with antimicrobial and the region that does not require the antimicrobial is made with non-porous materials.

In some embodiments, the antimicrobial is infused within the polymer before the polymer is formed into the oral appliance. In some embodiments, the oral appliance is immersed in the liquid antimicrobial and absorbs the liquid antimicrobial into the polymer. In other embodiments, the antimicrobial is preloaded into the hydrogel. The non-porous portions cannot absorb the antimicrobial and the antimicrobial cannot diffuse into the non-porous portions of the oral appliance.

In other embodiments, the entire oral appliance, the porous material, or discrete regions of the oral appliance includes or is manufactured to include carbon foam, polymer(s), or a combination thereof. The foam can be a carbon foam lattice, such as carbon resin DPR 10 (Carbon 3D, Inc. C. A.). These polymer materials can be printed, for example, by Carbon 3D printers.

The carbon foam, and/or polymer(s), can affect the application and/or release of the antimicrobial. In some embodiments, the entire oral appliance is made from carbon foam or polymer(s), and the density of the carbon foam or polymer(s) vary from very dense regions which create the non-porous regions of the oral appliance, to less dense regions which create the porous regions of the oral appliance. The very dense, non-porous regions prevent the release of the antimicrobial.

The carbon foam or polymer(s) allow the porosity of the oral appliance to be controlled. For example, the oral appliance can be 3D printed with carbon foam or polymer(s) and the areas not including the gum line can be printed densely where there are little to no open cell, lattice or honeycomb configurations present. However, the gum line area will be printed with the carbon foam or polymer(s) in a less dense manner where open cell, lattice or honeycomb configurations are present to allow influx, or allow release of antimicrobial or other substances to the gum line area. In some embodiments, some areas of the oral appliance can be printed virtually as a solid, and other areas of the oral appliance can be printed as a semi-solid.

In some embodiments, when the oral appliance is made by 3D printing and different polymers having a different density are used, the printer can be instructed to print the low-density polymer at discrete regions of the oral appliance, which will be the porous region of the oral appliance. The printer can be instructed to print a higher density of polymer on the oral appliance to make discrete non-porous regions of the oral appliance.

It is to be understood that the polymerizable liquid is reactive to irradiation such as light (e.g., ultraviolet (UV) light) and the polymerizable liquid can contain photoreactive or photocurable groups for such reactivity to take place. The UV light can be controlled by a computer and the light will irradiate the polymerizable liquid for polymerization. In some embodiments, curing can be initiated by heat, radiation, electron beams, or chemical additives. In other embodiments, curing can occur by thermo setting in the absence of additives.

After the oral appliance is made, in some embodiments, the antimicrobial in a polymer (e.g., hydrogel) can be disposed at discrete regions of the oral appliance in any manner This can be accomplished by manually placing the antimicrobial in the oral appliance at discrete regions of it. In some embodiments, the antimicrobial in a polymer (e.g., hydrogel) can be disposed at discrete regions of the oral appliance by machine.

These and other aspects of the present application will be further appreciated upon consideration of the following Examples, which are intended to illustrate particular embodiments of the application but are not intended to limit its scope, as defined by the claims.

### EXAMPLES

Examples 1 to 13 are directed to various HEMA hydrogel formulations. For these examples, materials and equipment were obtained as follows:
Analytical balance (Ohaus, +/−0.1 mg)
Rheometer (TA instruments AR2000)
UV lamp (Blak-Ray B100AP)
Mayonnaise (Hellmann' s)
Ethanol (200 proof, USP grade)
Eppendorf Pipettes/tips (VWR)
CaSO4 (blue indicating, Drierite)
1-Hydroxycyclohexyl phenyl ketone (Irgacure 184, Aldrich)
Hydroxyethlymethacrylate (HEMA, Aldrich)
Ethylene glycol dimethacrylate (EGDMA, Aldrich)
Poly(ethylene glycol) diacrylate (PEGDA, Aldrich)
Chlorhexidine digluconate solution (CHX, Aldrich)
Branson 5510 Sonicator (Branson)
Loctite UV-curing system with UV wand (Loctite)
HPLC system Waters 2695 Separation Module and Waters 996 photodiode array detector
(Synergi 4um 150×4.6 mm LC column, 40:60 Acetonitrile: 0.1% Trifluoracetic acid in DIH2O)
Hydroxypropylcellulose (Type HF PHARM, Klucel) (HPC 1.15M)
Poly(2-hydroxyethyl methacrylate) (300,000 Da, Aldrich) (pHEMA 300kDa)
Poly(2-hydroxyethyl methacrylate) (1,000,000 Da, Aldrich) (pHEMA 1M Da)

### Example 1 -30% pHEMA 300kDa

A method for preparing a hydrogel composition including 30% pHEMA 300kDa and 0.001% (v/v) EGDMA is provided. In a scintillation vial 4.4946 g of pHEMA 300,000 Da was weighed out and 15 mL of HEMA was added and was allowed to dissolve with overhead stirring each day and placed in a 37° C. incubator overnight until dissolution was complete. Once dissolved 1 mL of this solution was placed in a scintillation vial along with 0.010 mL of a stock solution (10%w/v EGDMA:HEMA) and 0.015 mL of an Irgacure 184 solution (0.1005 g of 1-hydroxycyclohexyl phenyl ketone dissolved in 1 mL of anhydrous DMSO) was added, physically stirred and then 0.5 mL of this solution was placed on a square piece of Rigid 0.75 material. Microscope pictures were taken at 0, 30, 60, 90, & 120 seconds. This was placed under a Loctite UV-curing system until crosslinking was observed. Crosslinking times, crosslinking observations and other synthesis observations are reported in Table 1 of this disclosure. Area at each time point was calculated as well as the area of hydrogel after UV curing and the results are shown in Table 2.

### Example 2 -30% pHEMA 300kDa

A method for preparing a hydrogel composition including 30% pHEMA 300k and 0.003% (v/v) EGDMA is provided. In a scintillation vial 4.4946 g of pHEMA 300,000 Da was weighed out and 15 mL of HEMA was added and was allowed to dissolve with overhead stirring each day and placed in a 37° C. incubator overnight until dissolution was complete. Once dissolved 1 mL of this solution was placed in a scintillation vial along with 0.030 mL of a stock solution (10%w/v EGDMA:HEMA) and 0.015 mL of an Irgacure 184 solution (0.1005 g of 1-hydroxycyclohexyl phenyl ketone dissolved in 1 mL of anhydrous DMSO) was added, physically stirred and then 0.5 mL of this solution was placed on a square piece of Rigid 0.75 material. Microscope pictures were taken at 0, 30, 60, 90, & 120 seconds. This was placed under a Loctite UV-curing system until crosslinking was observed. Crosslinking times, crosslinking observations and other synthesis observations are reported in Table 1. Area at each time point was calculated as well as the area of hydrogel after UV curing and the results are shown in Table 2.

### Example 3 -30% pHEMA 300kDa

A method for preparing a hydrogel composition including 30% pHEMA 300k and 0.006% (v/v) EGDMA is provided. In a scintillation vial 4.4946 g of pHEMA 300,000 Da was weighed out and 15 mL of HEMA was added and was allowed to dissolve with overhead stirring each day and placed in a 37° C. incubator overnight until dissolution was complete. Once dissolved 1 mL of this solution was placed in a scintillation vial along with 0.010 mL of a stock solution (10%w/v EGDMA:HEMA) and 0.015 mL of an Irgacure 184 solution (0.1005 g of 1-hydroxycyclohexyl phenyl ketone dissolved in 1 mL of anhydrous DMSO) was added, physically stirred and then 0.5 mL of this solution was placed on a square piece of rigid 0.75 material. Microscope pictures were taken at 0, 30, 60, 90, & 120 seconds. This was placed under a Loctite UV-curing system until crosslinking was observed. Crosslinking times, crosslinking observations and other synthesis observations are reported in Table 1. Area at each time point was calculated as well as the area of hydrogel after UV curing and the results are shown in Table 2.

### Example 4 -20% pHEMA 300kDa

A method for preparing a hydrogel composition including 20% pHEMA 300kDa and 0.001% (v/v) EGDMA is provided. In a scintillation vial 1.0078 g of pHEMA 300,000 Da was weighed out and 5 mL of HEMA was added and was allowed to dissolve with overhead stirring each day and placed in a 37° C. incubator overnight until dissolution was complete. Once dissolved 1 mL of this solution was placed in a scintillation vial along with 0.010 mL of a stock solution (10%w/v EGDMA:HEMA) and 0.015 mL of an Irgacure 184 solution (0.1005 g of 1-hydroxycyclohexyl phenyl ketone dissolved in 1 mL of anhydrous DMSO) was added, physically stirred and then 0.5 mL of this solution was placed on a square piece of Rigid 0.75 material. Microscope pictures were taken at 0, 30, 60, 90, & 120 seconds. This was placed under a Loctite UV-curing system until crosslinking was observed. Crosslinking times, crosslinking observations and other synthesis observations are reported in Table 1. Area at each time point was calculated as well as the area of hydrogel after UV curing and the results are shown in Table 2.

### Example 5 -20% pHEMA 300kDa

A method for preparing a hydrogel composition including 20% pHEMA 300kDa and 0.002% (v/v) EGDMA is provided. In a scintillation vial 1.0078 g of pHEMA 300,000 Da was weighed out and 5 mL of HEMA was added and was allowed to dissolve with overhead stirring each day and placed in a 37° C. incubator overnight until dissolution was complete. Once dissolved 1 mL of this solution was placed in a scintillation vial along with 0.020 mL of a stock solution (10%w/v EGDMA:HEMA) and 0.015 mL of an Irgacure 184 solution (0.1005 g of 1-hydroxycyclohexyl phenyl ketone dissolved in 1 mL of anhydrous DMSO) was added, physically stirred and then 0.5 mL of this solution was placed on a square piece of Rigid 0.75 material. Microscope pictures were taken at 0, 30, 60, 90, & 120 seconds. This was placed under a Loctite UV-curing system until crosslinking was observed. Crosslinking times, crosslinking observations and other synthesis observations are reported in Table 1. Area at each time point was calculated as well as the area of hydrogel after UV curing and the results are shown in Table 2.

### Example 6 -20% pHEMA 300kDa

A method for preparing a hydrogel composition including 20% pHEMA 300kDa 0.005% (v/v) EGDMA is provided. In a scintillation vial 1.0078 g of pHEMA 300,000 Da was weighed out and 5 mL of HEMA was added and was allowed to dissolve with overhead stirring each day and placed in a 37° C. incubator overnight until dissolution was complete. Once dissolved 1 mL of this solution was placed in a scintillation vial along with 0.050 mL of a stock solution (10%w/v EGDMA:HEMA) and 0.015 mL of an Irgacure 184 solution (0.1005 g of 1-hydroxycyclohexyl phenyl ketone dissolved in 1 mL of anhydrous DMSO) was added, physically stirred and then 0.5 mL of this solution was placed on a square piece of Rigid 0.75 material. Microscope pictures were taken at 0, 30, 60, 90, & 120 seconds. This was placed under a Loctite UV-curing system until crosslinking was observed. Crosslinking times, crosslinking observations and other synthesis observations are reported in Table 1. Area at each time point was calculated and the results are shown in Table 2.

### Example 7 -15% pHEMA 1M

A method of preparing 15% pHEMA 1M Da hydrogel is provided. In a scintillation vial 1.5201 g of pHEMA 1M Da was weighed out and 10 mL of HEMA was added and was allowed to dissolve with overhead stirring each day and placed in a 37° C. incubator overnight until dissolution was complete. Once dissolved, 3 mL of this solution was placed in a scintillation vial along with 0.030 mL of a stock solution (10%w/v EGDMA:HEMA) and 0.045 mL of an Irgacure 184 solution (0.1005 g of 1-hydroxycyclohexyl phenyl ketone dissolved in 1 mL of anhydrous DMSO) was added, physically stirred to incorporate. A large scoop of this solution was breech loaded in a 3 mL syringe equipped with a needle with the tip cut off and was inject onto tray matched to Jarret's top teeth and was placed under a Loctite UV-curing system until crosslinking was observed. The rest of this solution was placed on a square piece of Rigid 0.75 material. This was placed under a Loctite UV-curing system until crosslinking was observed.

### Example 8 -15% pHEMA 1M

A method of preparing 15% pHEMA 1M Da hydrogel is provided. In a scintillation vial 1.5201 g of pHEMA 1M Da was weighed out and 10 mL of HEMA was added and was allowed to dissolve with overhead stirring each day and placed in a 37° C. incubator overnight until dissolution was complete. Once dissolved, 5 mL of this solution was placed in a scintillation vial along with 0.050 mL of a stock solution (10%w/v EGDMA:HEMA) and 0.075 mL of an Irgacure 184 solution (0.1016 g of 1-hydroxycyclohexyl phenyl ketone dissolved in 1 mL of anhydrous DMSO) was added and physically stirred to incorporate it. Then 0.5 mL of this solution was placed on a square piece of Rigid 0.75 material. Microscope pictures were taken at 0, 30, 60, 90, & 120 seconds. This was repeated two more times to make three loading and release (LR) samples. These were placed under a Loctite UV-curing system until crosslinking was observed. The area at each time point was calculated and the results are shown in Table 2. Then 0.5 mL of this solution was placed on paraffin lined petri dish and crosslinked using a Loctite UV-curing system. This was repeated two more times to make three mechanical (M) samples. Crosslinking times, crosslinking observations and other synthesis observations are reported in Table 1 of this disclosure.

### Example 9 -3% HPC 1.15M

A method of preparing a 3% HPC 1.15M polymeric composition is provided. In a scintillation vial 0.5025 g of HPC 1.15M was weighed out and 15 mL of HEMA was added and was allowed to dissolve with overhead stirring each day and placed in a 37° C. incubator overnight until dissolution was complete. Once dissolved, 3 mL of this solution was placed in a scintillation vial along with 0.030 mL of a stock solution (10%w/v EGDMA:HEMA) and 0.045 mL of an Irgacure 184 solution (0.1016 g of 1-hydroxycyclohexyl phenyl ketone dissolved in 1 mL of anhydrous DMSO) was added and physically stirred to incorporate it. Then 0.5 mL of this solution was placed on a square piece of Rigid 0.75 material. Microscope pictures were taken at 0, 30, 60, 90, & 120 seconds. This was repeated two more times to make three loading and release (LR) samples. These were placed under a Loctite UV-curing system until crosslinking was observed. Crosslinking times, crosslinking observations and other synthesis observations are reported in Table 1. The area at each time point was calculated and is shown in Table 2. Then 0.5 mL of this solution was placed on a square piece of Rigid 0.75 material and crosslinked to make a sonication (S) sample; 0.5 mL of this solution was placed on paraffin lined petri dish and crosslinked using a Loctite UV-curing system to make one mechanical (M) sample.

### Example 10 -50% pHEMA 300kDa

A method of preparing a 50% pHEMA 300kDa hydrogel is provided. In a scintillation vial 7.500 g of pHEMA 300,000 Da was weighed out and 15 mL of HEMA was added and was allowed to dissolve with overhead stirring each day and placed in a 37° C. incubator overnight until dissolution was complete. Once dissolved, 5.0641 g of this solution was placed in a scintillation vial along with 0.050 mL of a stock solution (10%w/v EGDMA:HEMA) and 0.075 mL of an Irgacure 184 solution (0.1008 g of 1-hydroxycyclohexyl phenyl ketone dissolved in 1 mL of anhydrous DMSO) was added, physically stirred and then — 0.5 g of this solution was placed on a square piece of Rigid 0.75 material. Microscope pictures were taken at 0, 30, 60, 90, & 120 seconds and are shown in FIG. 5. This was repeated two more times to make three loading and release (LR) samples. These were placed under a Loctite UV-curing system until crosslinking was observed. Crosslinking times, crosslinking observations and other synthesis observations are reported in Table 1. The area at each time point was calculated and the results are shown in Table 2. Then 0.5 mL of this solution was placed on paraffin lined petri dish and crosslinked using a Loctite UV-curing system. This was repeated two more times to make three mechanical (M) samples.

### Example 11 -50% pHEMA 300kDa

A method of preparing 50% pHEMA 300kDa hydrogel using Irgacure curing agent is provided. In a scintillation vial 7.500 g of pHEMA 300,000 Da was weighed out and 15 mL of HEMA was added and was allowed to dissolve with overhead stirring each day and placed in a 37° C. incubator overnight until dissolution was complete. This should make a 1:5.4 (w/w) ratio of pHEMA: HEMA. Once dissolved, 2.0705g of this solution was placed in a scintillation vial along with 0.020 mL of a stock solution (10%w/v EGDMA:HEMA) and 0.1493g of Irgacure 184 (1-hydroxycyclohexyl phenyl ketone) was added, physically stirred and then — 0.5 g of this solution was placed on a square piece of Rigid 0.75 material. This was placed under a Loctite UV-curing system until crosslinking was observed. Crosslinking times, crosslinking observations and other synthesis observations are reported in Table 1.

### Example 12 -50% pHEMA 300kDa

A method of preparing 50% pHEMA 300kDa hydrogel using Irgacure curing agent is provided. In a scintillation vial 7.500 g of pHEMA 300,000 Da was weighed out and 15 mL of HEMA was added and was allowed to dissolve with overhead stirring each day and placed in a 37° C. incubator overnight until dissolution was complete. This should make a 1:5.4 (w/w) ratio of pHEMA: HEMA. Once dissolved 2.0085g of this solution was placed in a scintillation vial along with 0.020 mL of a stock solution (10%w/v EGDMA:HEMA) and 0.2984g of Irgacure 184 (1-hydroxycyclohexyl phenyl ketone) was added, physically stirred and then — 0.5 g of this solution was placed on a square piece of Rigid 0.75 material. This was placed under a Loctite UV-curing system until crosslinking was observed. Crosslinking times, crosslinking observations and other synthesis observations are reported in Table 1.

### Example 13 -3% HPC 1.15M Da

A method of preparing 3% HPC 1.15M polymeric composition is prepared. In a scintillation vial 0.5025 g of HPC 1.15M was weighed out and 15 mL of HEMA was added and was allowed to dissolve with overhead stirring each day and placed in a 37° C. incubator overnight until dissolution was complete. Once dissolved 2 mL of this solution was placed in a scintillation vial along with 0.020 mL of a stock solution (10%w/v EGDMA:HEMA) and 0.030 mL of an Irgacure 184 solution (0.1008 g of 1-hydroxycyclohexyl phenyl ketone dissolved in 1 mL of anhydrous DMSO) was added and physically stirred to incorporate it. Then 0.5 mL of this solution was placed on paraffin lined petri dish. This was repeated one more time to make two mechanical (M) samples. These were placed under a Loctite UV-curing system until crosslinking was observed. Crosslinking times, crosslinking observations and other synthesis observations are reported in Table 1.

Table 1 below indicates crosslinking time for Examples 1-6 and 8-12 discussed above. Table 2 below illustrates % increase of area after 30 seconds and 120 seconds of uncured hydrogels immediately after dispensing on a flat, level piece of base polymer for Examples 1-6 and 8-10 discussed above. The spreading of hydrogel indicates both the degree of interfacial tension of the hydrogel formulation against the base plastic as well as the viscosity and represents the degree of spread to be anticipated during manufacturing of these hydrogels.

TABLE 1

| | | | | All other general |
|---|---|---|---|---|
| | Crosslinking | | Synthesis/Crosslinking | observations during |
| Sample name | time | | observations | testing |
| Ex. 1 - 30% pHEMA | >2 | minutes | Standard amount of | |
| 300k 0.001% | | | EGDMA. Slightly less | |
| EGDMA LR | | | viscous than mayonnaise. | |
| Ex. 2 - 30% pHEMA | >2 | minutes | Did not seem to increase | No damage noted during |
| 300k 0.003% | | | viscosity vs. standard | testing. Takes the same |
| EGDMA LR | | | amount of EGDMA | amount of time to wash |
| | | | | out unreacted material |
| | | | | than standard amount of |
| | | | | EGDMA. |
| Ex. 3 - 30% pHEMA | >2 | minutes | Did not seem to increase | No damage noted during |
| 300k 0.006% | | | viscosity vs. standard | testing. Takes the same |
| EGDMA LR | | | amount of EGDMA | amount of time to wash |
| | | | | out unreacted material |
| | | | | than standard amount of |
| | | | | EGDMA. |
| Ex. 4 - 20% pHEMA | >2 | minutes | Standard amount of | |
| 300k 0.001% | | | EGDMA. Slightly less | |
| EGDMA LR | | | viscous than mayonnaise. | |
| Ex. 5 20% pHEMA | >2 | minutes | Did not seem to increase | No damage noted during |
| 300k 0.002% | | | viscosity vs. standard | testing. Takes the same |
| EGDMA LR | | | amount of EGDMA | amount of time to wash |
| | | | | out unreacted material |
| | | | | than standard amount of |
| | | | | EGDMA. |
| Ex. 6 - 20% pHEMA | >2 | minutes | Did not seem to increase | No damage noted during |
| 300k 0.005% | | | viscosity vs. standard | testing. Takes the same |
| EGDMA LR | | | amount of EGDMA | amount of time to wash |
| | | | | out unreacted material |
| | | | | than standard amount of |
| | | | | EGDMA. |
| Ex. 8 - 15% pHEMA | >2 | minutes | Slightly less viscous | |
| 1M LR | | | than mayonnaise and an | |
| | | | extremely stringy | |
| | | | solution. | |
| Ex. 9 - 3% HPC | >2 | minutes | Turns opaque during | Turns white upon |
| 1.15M LR | | | crosslinking | cleaning procedure |
| Ex. 10 - 50% pHEMA | 45 | sec | Extremely viscous and | |
| 300k LR | | | stringy solution. | |
| | | | Crosslinks faster than | |
| | | | 3% HPC 1.15M | |
| | | | formulation. Standard | |
| | | | amount of Irgacure | |
| Ex. 11 - 50% pHEMA | 20 | sec | 5 times the amount of | Takes much longer to |
| 300k and Irgacure | | | Irgacure than standard | clean out unreacted |
| | | | formulation. Slightly | material than standard |
| | | | faster crosslinking then | amount of Irgacure. This |
| | | | standard amount of | sample was not moved |
| | | | Irgacure. Turns white | forward from the |
| | | | upon crosslinking | washing step. Strong |
| | | | | odor noted coming from |
| | | | | washing water. |
| Ex. 12 - 50% pHEMA | 30 | sec | 10 times the amount of | Takes much longer to |
| 300k and Irgacure | | | Irgacure than standard | clean out unreacted |
| | | | formulation. Slightly | material than standard |
| | | | faster crosslinking then | amount of initiator. This |
| | | | standard amount of | sample was not moved |
| | | | Irgacure. Turns a | forward from the |
| | | | yellowish white upon | washing step. Strong |
| | | | crosslinking. | odor noted coming from |
| | | | | washing water. |

TABLE 2

| | % increase of | % increase of |
|---|---|---|
| | area after 30 | area after 120 |
| Sample name | sec | sec |
| Ex. 1 - 30% pHEMA 300k 0.001% | 24.92 | 52.00 |
| EGDMA LR (n = 1) | | |
| Ex. 2 - 30% pHEMA 300k 0.003% | 24.98 | 43.61 |
| EGDMA LR (n = 1) | | |
| Ex. 3 - 30% pHEMA 300k 0.006% | 26.03 | 49.67 |
| EGDMA LR (n = 1) | | |
| Ex. 4 - 20% pHEMA 300k 0.001% | 42.09 | 76.46 |
| EGDMA LR (n = 1) | | |
| Ex. 5 - 20% pHEMA 300k 0.002% | 35.30 | 62.83 |
| EGDMA LR (n = 1) | | |
| Ex. 6 - 20% pHEMA 300k 0.005% | 35.31 | 53.03 |
| EGDMA LR (n = 1) | | |
| Ex. 8 - 15% pHEMA 1M LR (n = 3) | 44.63 ± 24.26 | 80.99 ± 22.80 |
| Ex. 9-3% HPC 1.15M LR (n = 3) | 45.07 ± 10.55 | 82.44 ± 22.19 |
| Ex. 10 - 50% pHEMA 300k (n = 1) | 16.94 | 36.22 |

As illustrated in Table 2, the percent increase of area after dispensing uncured hydrogel onto a flat piece of polymer varies (i) from about 42.09 to about 24.92 after 30 seconds for hydrogel comprising pHEMA and EGDMA; (ii) from about 76.67 to about 43.61 after 120 seconds for hydrogel comprising pHEMA and EGDMA; (iii) from about 16.94 to about 4.63 +/−24.26 for hydrogel comprising pHEMA after 30 seconds; (iv) from about 36.22 to about 80.99 +/−22.80 after 60 seconds; (v) about 45.07 +/−10.55 for hydrogel comprising HPC after 30 seconds; or about 82.44 +/−22.19 for hydrogel comprising HPC after 60 seconds.

### Example 14

In this Example, the swelling and hydration of hydrogel samples prepared according to Examples 8, 9 and 10 are examined The theoretical chlorhexidine digluconate uptake of these samples is also examined

Samples of 15% pHEMA 1M LR (example 8) , 3% HPC 1.15M LR (example 9), and 50% pHEMA 300k LR (example 10) were soaked upside-down in 10 ml deionized water (DI) water at 37° C. overnight and weighed at predetermined time periods until hydrogels no longer gained weight. All hydrogels were left in the 37° C. incubator soaking upside-down in 10 mL of water and weighed the next morning to make sure they were fully hydrated. Percent weight increase was calculated and is in the results section under Table 4 below.

TABLE 4

| | | | Theoretical |
|---|---|---|---|
| | Time for | | chlorhexidine |
| | 0.5 g of | | digluconate uptake |
| | dry hydrogel | % weight | of 0.2 mL of |
| | to fully | increase | dispensed hydrogel |
| | hydrate | after | soaked in a 1% |
| Sample name | (hr) | hydration | solution (mg) |
| 15% pHEMA 1M | 18 (n = 1) | 58.36 ± 6.04 | 1.13 |
| 190812FAJ-A139 | | | |
| LR (n = 3) | | | |
| 3% HPC 1.15M | 19 (n = 1) | 43.26 ± 14.09 | 1.00 |
| 190812FAJ-B139 | | | |
| LR (n = 3) | | | |
| 50% pHEMA 300k | 24 (n = 1) | 61.95 ± 5.04 | 1.42 |
| 190829FAJ-A139 | | | |
| LR (n = 3) | | | |

The theoretical chlorhexidine digluconate uptake in a 0.2 mL dispensed hydrogel soaked using a 1% chlorhexidine digluconate solution was calculated based on a density for pHEMA of 1.15 g/mL. Given the density of pHEMA is 1.15 g/mL, a sample of 0.2 mL of dispensed hydrogel would have the weight of 0.23 g. Assuming the density of 1% chlorhexidine digluconate solution to be equal to pure water of 1 g/ml, the theoretical chlorhexidine digluconate uptake can be calculated as (((0.23×(% weight increase of specified formulation after hydration))−0.23)×(percent w/v of chlorhexidine digluconate solution).

As illustrated in Table 4, a hydrogel comprising (i) 15% pHEMA 1M has 58.36 +/−6.04% weight increase after hydration; (ii) 3% HPC 1.15M has 43.26 +/−14.09% weight increase after hydration; or (iii) 50% pHEMA 300 kDa has 61.95 +/−5.04% weight increase after hydration. As further illustrated in Table 4, 0.2 mL of fully hydrated hydrogel having (i) 15% pHEMA 1M has an uptake of chlorhexidine gluconate of about 1.13 mg; (ii) 3% HPC 1.15M has an uptake of chlorhexidine gluconate of about 1.00 mg; or (iii) 50% pHEMA 300 kDa has an uptake of chlorhexidine gluconate of about 1.42 mg.

### Example 15

In this Example samples prepared in Examples 8 and 9 were subjected to washing tests followed by UV-visible tests of reactants and washed samples.

Samples 3% HPC 1.15M (Example 9) and 15% pHEMA 1M LR3 (Example 8) were hydrated in 10 mL of deionized H₂O (DiH₂O) and placed in 37° C. incubator without orbital shaking overnight. These samples were then sonicated for 10 minutes intervals with fresh 10 mL of DiH₂O in each interval. UV Visible (UV Vis) scans were generated after every 4th wash until peaks were less than 0.2 ABS. Table 3 below illustrates results of this sonication after hydration method assay.

TABLE 3

| | mL of DI | Number of 10-minute |
|---|---|---|
| | H₂O each | sonications before |
| Sample name | sonication | sample was deemed clean |
| 15% pHEMA 1M LR3 (Ex. 8) | 10 | 40 |
| 3% HPC 1.15M (Ex. 9)_{—} | 10 | 60 |

UV visible scans between 190 and 900 nm of reactants in solution were generated. UV Vis scans between 190 and 900 nm of hydrogel washing water indicates the degree of success of the removal of unreacted material from cured hydrogels.

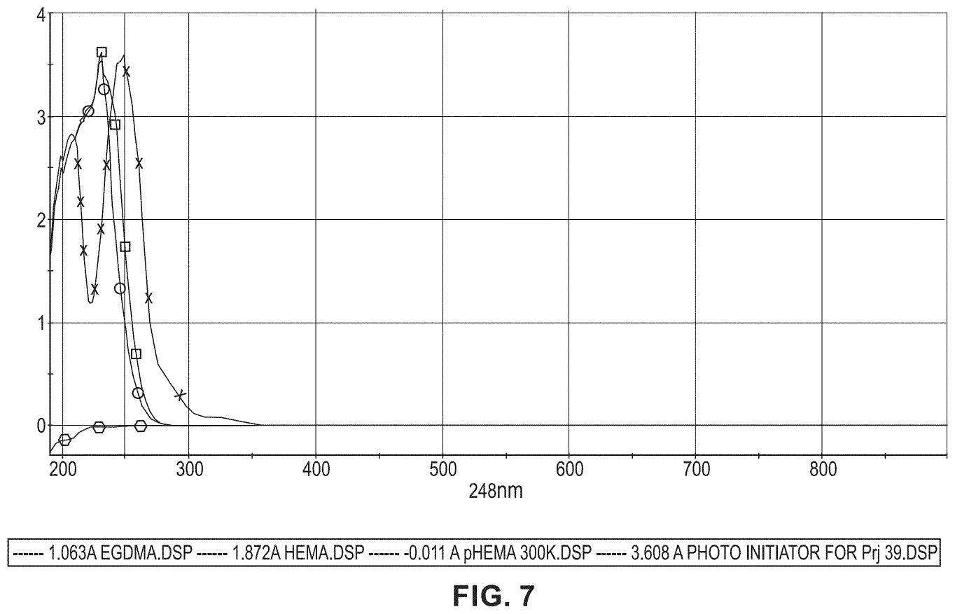

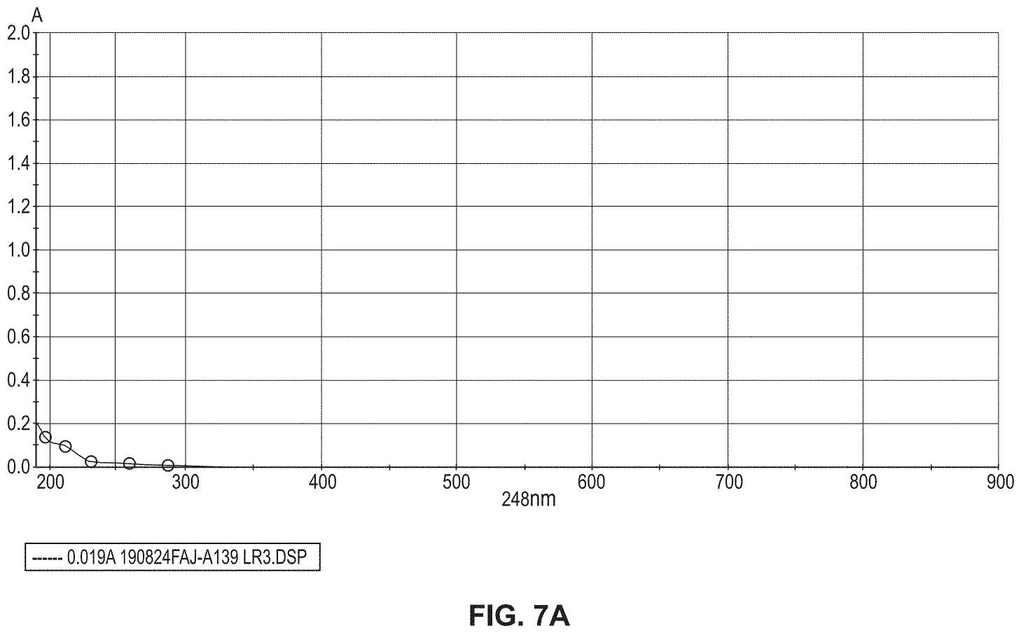

In a series of scintillation vials a small amount of each reactant used in the generation of hydrogels was placed and 10 mL of DiH2O was added to dissolve. These were placed in a 37° C. orbital incubator for 4 hours. Two milliliters of each solution was used to generate UV Vis scans from 190 nm to 900 nm. Irgacure 184 had two peaks, one at 206 nm and the another at 248 nm, EGDMA and HEMA had an absorbance at 230 nm. pHEMA had no absorbance and HPC was not tested due to its hydrophobicity and its relative low toxicity. The results are illustrated in FIGS. 7 and 7A of this disclosure. FIG. 7 shows overlaid UV visible scans of hydrogel reactants. EGDMA is represented by a curve having circles; HEMA is represented by a curve having squares; pHEMA is represented by a curve having hexagons and the photoinitiator Irgacure is represented by a curve having x signs. The vertical line at 248 nm represents absorbance. FIG. 7A is an overlaid UV visible scan of a loading and release (LR) sample of 50% pHEMA 300kDa hydrogel as prepared in Example 10 cleared of the washing procedure. As in FIG. 7, the vertical line represents absorbance at 248 nm.

All other hydrogels were washed by orbital agitation at 37° C/100 RPM in a shaking incubator (Southwest Science) in 100 ml of deionized (DI) water. At predetermined time points water was removed and replaced with fresh DI water. Then DI H₂O was tested by UV-Vis analysis against fresh DI water as a blank and continued washing until ABS below 0.2A after 200 nm. Water washes were tested by scanning on a UV/Vis Genesys 10S instrument in a quartz cuvette from 190-900 nm at 2 nm intervals against a deionized water blank. Examples of hydrogels that were considered clean enough to move on to the next procedure are illustrated in FIG. 7.

### Example 16

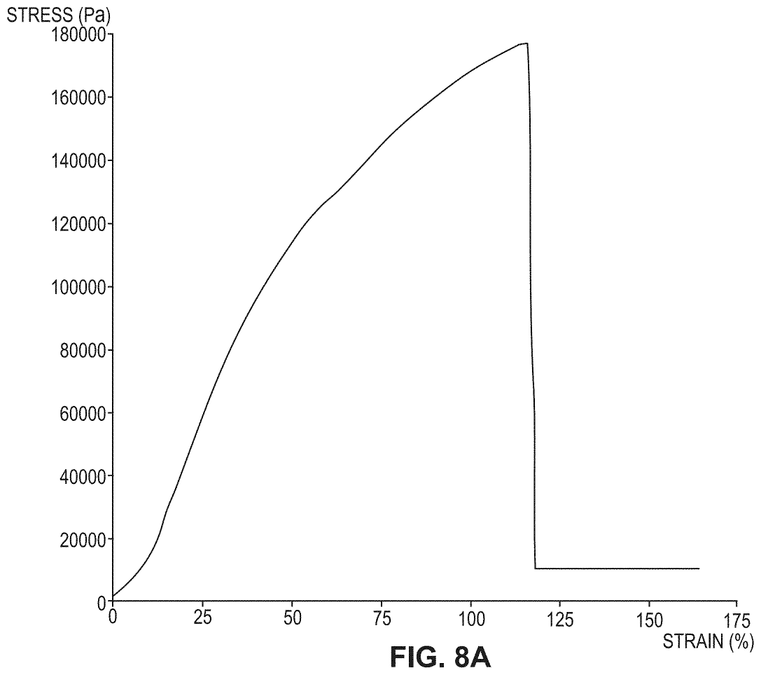

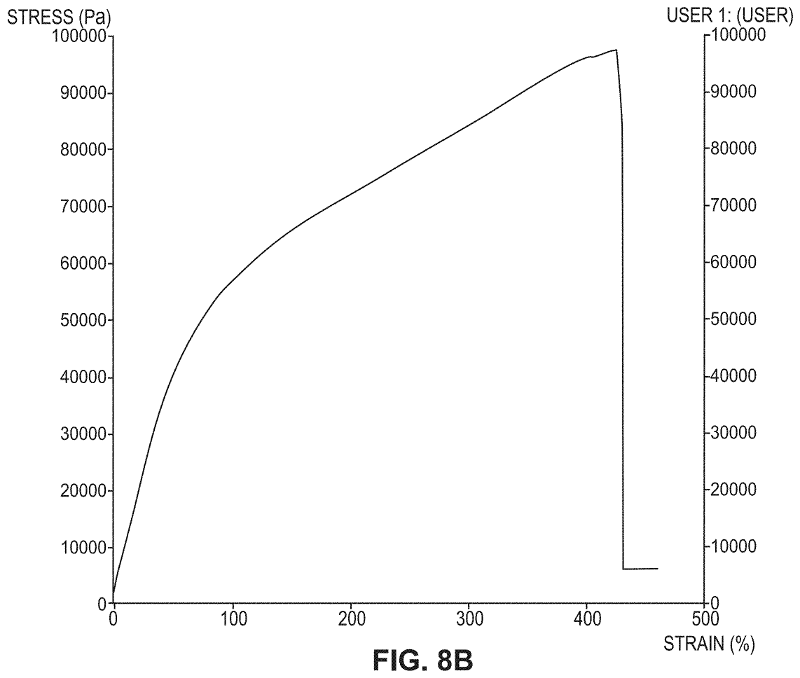

In this example, mechanical testing was performed on hydrated samples using a TA.XT Plus system equipped with a 5 kG load cell and tensile clips. Briefly, samples were cut to tensile-test dog bone shape and the cross-sectional area (width x thickness) was determined in mm using digital calipers (VWR). Each test sample was clamped into the system and drawn apart at a crosshead speed of 1 mm/sec until the material ruptured. Data analysis was performed using Exponent (Stable Microsystems) software. The mechanical stress at rupture was recorded as yield strength in Pascals and converted to either kPa or MPa as appropriate for range. The percent strain at point of rupture was recorded as extensibility. The slope of the stress-strain curve between 1-3% was determined to obtain the elastic modulus. Examples of graphs produced by the TA.XT Plus texture analyzer are shown in FIGS. 8A and 8B. Results generated by the mechanical tests conducted on TA.XT

Plus texture analyzer for indicated samples are summarized in Table 5.

TABLE 5

| | | | Tensile |
|---|---|---|---|
| | Elastic Modules | Elongation | strength |
| Sample name | (%/kPa) | (%) | (kPa) |
| 15% pHEMA 1M (Ex. 8) | 0.736 ± 0.285 | 431.1 ± 51.6 | 131 ± 30.6 |
| (n = 3) | | | |
| 3% HPC 1.15M (Ex. 9) | 1.5 ± 0.3 | 92.1 ± 32.9 | 124.9 ± 47.1 |
| (n = 3) | | | |
| 50% pHEMA 300 kDa | 0.541 ± 0.285 | 431.1 ± 51.6 | 109.5 ± 0.7 |
| (Ex. 10) (n = 3) | | | |
| 50% pHEMA 300k and | 0.134 | 66.1 | 4.0 |
| Irgacure (Ex. 11) | | | |
| (n = 1) | | | |
| 50% pHEMA 300 kDa | 0.266 | 742.9 | 10.3 |
| and Irgacure (Ex. 12) | | | |
| (n = 1) | | | |

As illustrated in the Table 5, the elastic modulus of the hydrogel useful in the oral appliance of this disclosure varies from about 0.27%/kPa to about 1.5 +/−0.3%/kPa. In various embodiments, the hydrogel comprising (i) 15% pHEMA has an elastic modulus of about 0.736 +/−0.285%/kPa; (ii) 3% HPC has an elastic modulus of about 1.5 +/−0.3%/kPa; (iii) 50% pHEMA has an elastic modulus of about 0.541 +/−0.285%/kPa; (iv) 50% pHEMA and 5x hydroxycyclohexyl phenyl ketone has an elastic modulus of about 0.134%/kPa ; and (v) 50% pHEMA and 10× 1-hydroxycyclohexyl phenyl ketone has an elastic modulus of about 0.266%/kPa.

In some of the embodiments illustrated in Table 5, the tensile strength of the hydrogel useful in the oral appliance of this disclosure varies from about 4.0 to about 131 kPa. In various embodiments, the hydrogel comprising (i) 15% pHEMA has a tensile strength of about 131 +/−30.6 kPa; (ii) 3% HPC has a tensile strength of about 124.9 +/−47.1 kPa; (iii) 50% pHEMA has a tensile strength of about 109.5 +/−0.7 kPa; (iv) 50% pHEMA and 5x hydroxycyclohexyl phenyl ketone has a tensile strength of about 4.0 kPa ; and (v) 50% pHEMA and 10x 1-hydroxycyclohexyl phenyl ketone has a tensile strength of about 10.3 kPa.

### Example 17

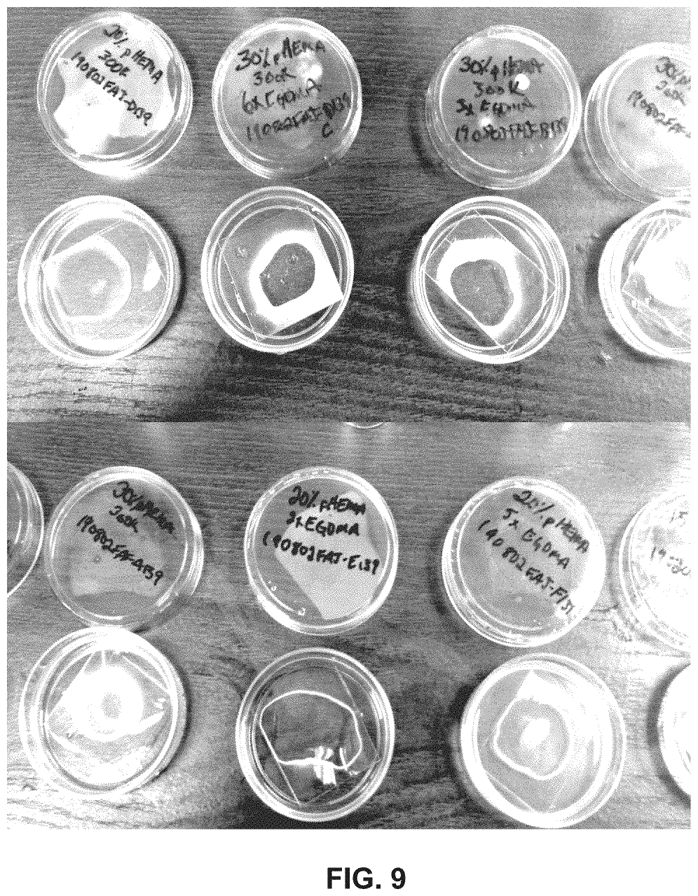

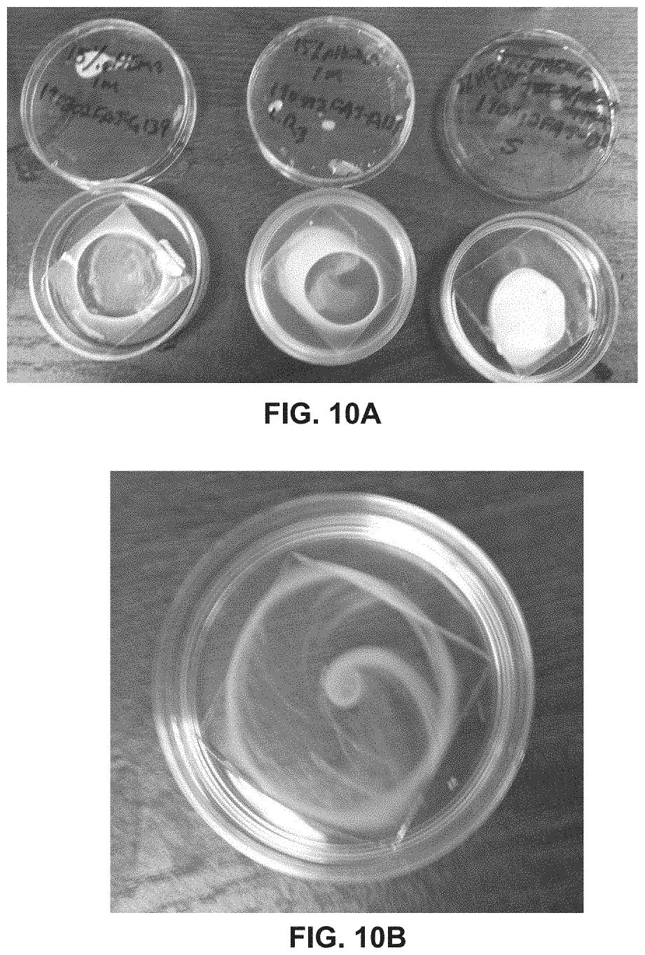

In this Example, 5% chlorhexidine digluconate loading and release at 37° C. was considered. For the hydrated and washed samples of 30% pHEMA 300kDa 0.001% (v/v) EGDMA (Ex. 1), 30% pHEMA 300kDa 0.003% (v/v) EGDMA (Ex. 2), 30% pHEMA 300kDa 0.006% (v/v) EGDMA (Ex. 3), 20% pHEMA 300kDa (Ex. 4), 20% pHEMA 300kDa 0.002% (v/v) EGDMA(Ex. 5), 20% pHEMA 300kDa 0.005% (v/v) EGDMA (ex. 6), 15% pHEMA 1M (Ex. 7), 15% pHEMA 1M LR (Ex. 8), and 3% HPC 1.15M S (Ex. 9), 5 mL of 5% chlorhexidine digluconate solution was placed in a 60 mm petri dish and the hydrogel samples were placed in the solution upside-down and allowed to soak for 30 minutes. Then, the hydrogel samples were transferred to another 60 mm petri dish and 10 mL of artificial saliva at 37 ° C. was added. After each time points of 10, 20, 30 minutes and after overnight the 10 ml of saliva was refreshed. Pictures of several samples are shown in FIG. 9. It is expected that other polymers can be used at 5% w/w or v/v or higher concentrations of chlorhexidine digluconate in different oral appliance constructions.

FIG. 9 shows pictures of hydrogel samples taken in loading and release assay. In particular, FIG. 9 illustrates pictures of 20% pHEMA hydrogels as prepared in Examples 4, 5 and 6; 30% pHEMA 300kDa hydrogels as prepared in Examples 1, 2, 3 and 14; and 15% pHEMA 1M as prepared in Example 7, during treatment with 5% chlorhexidine gluconate as prepared in Example 17. Below each hydrogel sample, there is a petri dish where each hydrogel was tested on a square piece of Rigid 0.75 material which was placed under a Loctite UV-curing system until crosslinking was observed.

### Example 18

In this Example, 1% chlorhexidine digluconate loading and release at 37° C. was measured using HPLC analysis.

For the samples of 15% pHEMA 1M LR (Ex. 8), 3% HPC 1.15M LR (Ex. 9) and 50% pHEMA 300kDa LR (Ex. 10), 5 mL of 1% chlorhexidine digluconate solution was placed in a 60 mm petri dish and the hydrogel samples were placed in the solution upside-down and allowed to soak for 30 minutes. Then the hydrogel samples were transferred into 5 mL of artificial saliva at 37 ° C. for predetermined time points. After time points of 10, 20, 30 minutes and overnight, the 5 ml of saliva was removed for HPLC testing of chlorhexidine digluconate content and refreshed with 5 more mL of artificial saliva.

HPLC analysis done using a HPLC system consisting of a Waters 2695 Separation Module and Waters 996 photodiode array detector was set up with Synergi 4um 150 x 4.6 mm LC column and flushed with 40:60 Acetonitrile: 0.1% Trifluoracetic acid in DIH₂O. Detection was set at 254 nm and a series of calibration standards were generated by serial diluting a stock 20% w/v chlorhexidine gluconate solution to 1%, 0.5%, 0.1%, 0.01%, 0.001% and 0.0001% w/v. The results for this analysis are reported in Table 6 below.

TABLE 6

| | | | | Total | Theoretical |
|---|---|---|---|---|---|
| | | | | chlorhexidine | chlorhexidine |
| | | | | gluconate | gluconate |
| | | | | amount released | uptake in ~0.5 g |
| | | 20 min | 30 min | by hydrogel | of dry hydrogel |
| | 10 min | release (mg) | release (mg) | (cumulative, | soaked in a 1% |
| Sample name | release (mg) | cumulative | cumulative | 12-16 hrs) (mg) | solution (mg) |
| 15% pHEMA 1M | 0.145 ± 0.097 | 0.147 ± 0.097 | 0.147 ± 0.097 | 0.164 ± 0.101 | 5.84 |
| 190812FAJ-A139 LR | | | | | |
| (n = 2) | | | | | |
| 3% HPC 1.15M | 0.003 ± 0.004 | 0.004 ± 0.004 | 0.004 ± 0.004 | 0.023 ± 0.011 | 4.33 |
| 190812FAJ-B139 LR | | | | | |
| (n = 3) | | | | | |
| 50% pHEMA 300k | 0.024 ± 0.007 | 0.026 ± 0.009 | 0.027 ± 0.008 | 0.033 ± 0.011 | 6.19 |
| 190829FAJ-A139 LR | | | | | |
| (n = 3) | | | | | |

As illustrated in the Table 6, the uptake amount of about 0.5 g of dry hydrogel soaked in a 1% solution of chlorhexidine gluconate of an oral appliance comprising (i) 15% pHEMA 1M is about 5.84 mg; (ii) 3% HPC 1.15M is about 4.33 mg; or (iii) 50% pHEMA having a molecular weight of 300 kDa is about 6.19 mg.

In some embodiments, the release of 1% of chlorhexidine gluconate from about 0.5 g of hydrogel soaked in a 1% solution of chlorhexidine gluconate comprising (i) 15% pHEMA 1M is about 0.147 +/−0.097 mg over a time range from about 10 to about 30 min; (ii) 3% HPC 1.15M is about 0.004 +/−0.004 mg over a time range from about 10 to about 30 min; or (iii) 50% pHEMA having a molecular weight of 300 kDa is about 0.027 +/−0.008 mg over a time range from about 10 to about 30 min

In other embodiments, the release of 1% of chlorhexidine gluconate from about 0.5 g of hydrogel soaked in a 1% solution of chlorhexidine gluconate comprising (i) 15% pHEMA 1M is about 0.164 +/−0.101 mg over a time range from about 12 to about 16 hrs; (ii) 3% HPC 1.15M is about 0.023 +/−0.011 mg over a time range from about 12 to about 16 hrs; or (iii) 50% pHEMA having a molecular weight of 300 kDa is about 0.033 +/−0.011 mg over a time range from about 12 to about 16 hrs.

### Example 19

This Example illustrates the curing of an oral appliance prepared for an actual patient. 3 mL of 15% pHEMA 1M in HEMA solution was placed in a scintillation vial and 0.03 mL of a stock solution (10% v/v EGDMA/HEMA) was stirred in along with 0.045 mL of Irgacure 184 solution (0.1005 g of Irgacure 184 dissolved in 1 mL DMSO) and was stirred to incorporate it. This solution was then breech loaded in a 3 mL syringe and a needle with the tip cut off was attached. This was then injected onto the gum line ridge of the tray matched to the teeth of a biodental sample. This was then held under the Loctite UV curing system wand until crosslinking was complete. After curing, the oral appliance was washed in 1.5 L of deionized water with shaking at room temperature for one hour then the water was refreshed and allowed to shake for another 1.5 hours. The oral appliance was then placed in the refrigerator over the weekend and removed and allowed to soak in 1L of deionized water for another hour. Subsequently, the oral appliance was removed from deionized water and was deemed ready for shipping to a potential user.

### Example 20

In this Example premixed kits for preparing the oral appliances described in this disclosure are provided. 0.9001 g of HPC (Hydroxypropylcellulose Type HF PHARM, Klucel) were mixed with 30 mL of HEMA by overhead stirring at room temperature until dissolved, placing the container in a 37° C. shaking incubator overnight until the next morning when stirring continued. Once dissolved, 0.03 mL EGDMA was added and stirred to incorporate it. A separate bottle of 1 gram of Irgacure 184 (1-Hydroxycyclohexyl phenyl ketone, Aldrich) was prepared. Both containers were enclosed and prepared for shipping.

The above Examples illustrate that as synthesized, the pHEMA hydrogels which are generated effectively with no solvent are in fully dry state. Hydrating the hydrogels until they have reached equilibrium can take up to 24 hours which is important for cleaning and loading afterwards as this opens up the hydrogel structure to allow contaminants to wash out and the antimicrobials to be loaded in.

Increasing the amount of photo initiator (Irgacure) was examined. This was found to speed up the crosslinking process only slightly, a few seconds, at most. However, it has proven to take much longer to clean the hydrogel after crosslinking and it is suspected that a lot of unreacted initiator seeps out of the hydrogel during the cleaning process. These results indicate that there is little benefit to adding photoinitiator in addition to the amount required for cros slinking.

Mechanical testing of each formulation was done and generally indicated these formulations were flexible and mechanically robust enough for the intended application. The incorporation of further photoinitator was observed to reduce the tensile strength.

As important as the cleanliness of the hydrogel is, the identity of the remaining component is also critical. Previous testing (primarily as it applies to contact lens applications) has demonstrated the biocompatibility of reacted poly(HEMA). Additionally, hydroxypropyl cellulose is listed as Generally Regards as Safe (GRAS) by FDA. This indicates that the primarily toxic components from the developed hydrogels are unreacted HEMA and EGDMA monomers as well as residual photoinitiator. All of these exhibit absorbance peaks at about 230 nm and above (248 nm for Irgacure) indicating that the UV-Vis absorbance specification for washing should be specified based on wavelength. If no peaks are above 0.2 A after 200 nm is observed, then the hydrogel is deemed cleaned and cleared to move forward in testing.

HPLC assays were done on hydrogels after they were soaked in a 1% chlorhexidine gluconate solution to examine the loading and release efficiency of different formulations. All formulations had a lower actual loading and release than what was to be expected based on theoretical assessments due to incomplete uptake during exposure. 15% pHEMA 1M had the best loading and release. However, this solution was not quit as viscous as mayonnaise and is a very stringy solution and makes it difficult to handle. 50% pHEMA had the second-best profile, however, the stringiness was also a problem with this solution. 3% HPC 1.15M had the lowest loading and release but had the best viscosity and the stringiness did not prove to be a problem when handling it, which proved to be the ideal solution to use in a manufacturing setting. Loading can be improved by allowing longer exposure times and modifying chlorhexidine gluconate concentration in the loading solution (at concentrations below that which can damage the base polymer) and this may be work of further studies at a later time. Notably, chlorhexidine gluconate may be effective even at very low concentrations so there may not be a need to load very high quantities.

While particular embodiments of the present disclosure have been shown and described, it will be appreciated by those skilled in the art that, based upon the teachings herein, changes and modifications may be made without departing from this disclosure and its broader aspects and, therefore, the appended claims are to encompass within their scope all such changes and modifications as are within the true spirit and scope of this disclosure. The true spirit and scope is considered to encompass devices and processes, unless specifically limited to distinguish from known subject matter, which provide equivalent functions as required for interaction with other elements of the claims and the scope is not considered limited to devices and functions currently in existence where future developments may supplant usage of currently available devices and processes yet provide the functioning required for interaction with other claim elements.

1. An oral appliance for delivering an antimicrobial to an oral cavity, the oral appliance having an exterior surface and an interior surface, the interior surface of the oral appliance configured to contour at least a portion of teeth and/or soft tissue areas of the oral cavity; and the antimicrobial dispensed in a carrier at discrete regions of the interior surface, the exterior surface or both the interior surface and the exterior surface of the oral appliance for delivering the antimicrobial to the oral cavity, wherein the antimicrobial is present in the carrier in an amount of about 0.01% w/w, v/v or w/v to about 20% w/w, v/v or w/v based on a total weight or a total volume of the carrier.

2. The oral appliance of claim 1, wherein the antimicrobial is chlorhexidine.

3. The oral appliance of claim 2, wherein the antimicrobial is chlorhexidine gluconate.

4. (canceled)

5. (canceled)

6. (canceled)

7. The oral appliance of claim 1, wherein the carrier comprises a hydrogel, which comprises about 10% to about 99% w/w, v/v or w/v of the carrier.

8. The oral appliance of claim 4, wherein the hydrogel comprises ethylene glycol dimethacrylate (EGDMA), polyvinyl alcohol (PVA), hydroxypropylcellulose (HPC) or poly(2-hydroxyethyl methacrylate) (pHEMA) or a combination thereof.

9. (canceled)

10. (canceled)

11. (canceled)

12. (canceled)

13. The oral appliance of claim 8, wherein the hydrogel has an elastic modulus from about 0.27%/kPa to about 1.5 +/−0.3%/kPa.

14. The oral appliance of claim 8, wherein the hydrogel has a tensile strength from about 4.0 to about 131 kPa.

15. (canceled)

16. A method of making an oral appliance for delivering an antimicrobial to an oral cavity, the method comprising providing an oral appliance having an exterior surface and an interior surface, the interior surface of the oral appliance configured to contour at least a portion of teeth and/or soft tissue areas of the oral cavity; and dispensing the antimicrobial in a carrier at discrete regions of the interior surface, the exterior surface or both the interior surface and the exterior surface of the oral appliance for delivering the antimicrobial to the oral cavity, wherein the antimicrobial is present in the carrier in an amount of about 0.01% to about 20 wt. % w/w, v/v or w/v based on a total weight or a total volume of the carrier.

17. The method of claim 16, wherein the antimicrobial is chlorhexidine.

18. The method of claim 17, wherein the chlorhexidine is chlorhexidine digluconate.

19. (canceled)

20. The method of claim 17, wherein the carrier comprises a hydrogel comprising polyvinyl alcohol (PVA).

21. (canceled)

22. (canceled)

23. (canceled)

24. The method of claim 20, wherein the hydrogel comprises about 10% to about 99% w/w, v/v or w/v of the carrier.

25. (canceled)

26. (canceled)

27. (canceled)

28. The method of claim 20, wherein the hydrogel has a crosslinking time from about 20 seconds to about greater than 2 minutes.

29. The method of claim 20, further comprising hydrating the hydrogel until the hydrogel is fully hydrated in about 18 hours to about 24 hours.

30. (canceled)

31. A method of treating an infected tissue of an oral cavity, the method comprising providing an oral appliance for delivering an antimicrobial to the infected tissue of the oral cavity, the oral appliance having an exterior surface and an interior surface, the interior surface configured to contour at least a portion of teeth and/or soft tissue areas of the oral cavity, the interior surface of the oral appliance having the antimicrobial in a carrier disposed at a discrete regions of the interior surface, the exterior surface or both the interior and exterior surface of the oral appliance for delivering the antimicrobial to the infected tissue, wherein the antimicrobial is present in the carrier in an amount of about 0.025% to about 20% w/w, v/v or w/v based on a total weight or a total volume of the carrier.

32. (canceled)

33. (canceled)

34. (canceled)

35. The method of claim 31, wherein the infected tissue is periodontal tissue.

36. The method of claim 31, wherein the antimicrobial is chlorhexidine gluconate.

37. (canceled)

38. The method of claim 31, wherein the carrier comprises a hydrogel comprising polyvinyl alcohol (PVA).

39. (canceled)

40. (canceled)

41. (canceled)

42. (canceled)

43. An antimicrobial composition for an oral appliance, the antimicrobial composition comprising chlorhexidine in a carrier, the chlorhexidine in an amount of about 0.001% to about 20% w/w, v/v or w/v based on a total weight or a total volume of the composition, and the carrier comprising at least one of polyvinyl alcohol (PVA), hydroxyethlymethacrylate (HEMA) ethylene glycol dimethacrylate (EGDMA), hydroxypropylcellulose (HPC) or poly(2-hydroxyethyl methacrylate) (pHEMA) or a combination thereof in an amount of from about 10% to about 99% w/w, v/v or w/v based on a total weight or a total volume of the composition.

44. The antimicrobial composition of claim 43, wherein the antimicrobial is chlorhexidine gluconate.

45. (canceled)

46. (canceled)
